(19) United States
(12) Patent Application Publication
(10) Pub. No.: US 2005/0059710 A1
(43) Pub. Date: Mar. 17, 2005

(54) Diphenylamino ketone derivatives as MEK inhibitors

(75) Inventors: Stephen Douglas Barrett, Hartland, MI (US); Michael David Kaufman, Ypsilanti, MI (US); Mark Stephen Plummer, Dexter, MI (US); Jessica Elizabeth Reed, Ann Arbor, MI (US); Bruce David Roth, Plymouth, MI (US); Kevin Matthew Schlosser, Ann Arbor, MI (US)

Correspondence Address: AGOURON PHARMACEUTICALS, INC., 10350 NORTH TORREY PINES ROAD, LA JOLLA, CA, 92037, US

(73) Assignee: Pfizer Inc

(21) Appl. No.: 10/896,514

(22) Filed: Jul. 22, 2004

Related U.S. Application Data
(60) Provisional application No. 60/489,599, filed on Jul. 23, 2003.

(51) Int. Cl. C07D 213/46; A61K 31/44; A61K 31/401; A61K 31/277; A61K 31/135

(52) U.S. Cl. 514/355; 514/524; 514/658; 546/315; 548/530; 558/410; 564/433; 514/423

(57) ABSTRACT

The present invention relates to diphenylamino ketone derivatives, pharmaceutical compositions and methods of use thereof.

This application claims the benefit of U.S. Provisional Application Ser. No. 60/489,599, filed Jul. 23, 2003, which is hereby incorporated by reference in its entirety.

### FIELD OF THE INVENTION

The present invention relates to diphenylamino ketone derivatives, pharmaceutical compositions and methods of use thereof.

### BACKGROUND OF THE INVENTION

MAPK/ERK Kinase (“MEK”) enzymes are dual specificity kinases involved in, for example, immunomodulation, inflammation, and proliferative diseases such as cancer and restenosis.

Proliferative diseases are caused by a defect in the intracellular signaling system, or the signal transduction mechanism of certain proteins. Defects include a change either in the intrinsic activity or in the cellular concentration of one or more signaling proteins in the signaling cascade. The cell may produce a growth factor that binds to its own receptors, resulting in an autocrine loop, which continually stimulates proliferation. Mutations or overexpression of intracellular signaling proteins can lead to spurious mitogenic signals within the cell. Some of the most common mutations occur in genes encoding the protein known as Ras, a G-protein that is activated when bound to GTP, and inactivated when bound to GDP. The above-mentioned growth factor receptors, and many other mitogenic receptors, when activated, lead to Ras being converted from the GDP-bound state to the GTP-bound state. This signal is an absolute prerequisite for proliferation in most cell types. Defects in this signaling system, especially in the deactivation of the Ras-GTP complex, are common in cancers, and lead to the signaling cascade below Ras being chronically activated.

Activated Ras leads in turn to the activation of a cascade of serine/threonine kinases. One of the groups of kinases known to require an active Ras-GTP for its own activation is the Raf family. These in turn activate MEK (e.g., MEK₁ and MEK₂) which then activates the MAP kinase, ERK (ERK₁ and ERK₂). Activation of MAP kinase by mitogens appears to be essential for proliferation; constitutive activation of this kinase is sufficient to induce cellular transformation. Blockade of downstream Ras signaling, for example by use of a dominant negative Raf-1 protein, can completely inhibit mitogenesis, whether induced from cell surface receptors or from oncogenic Ras mutants. Although Ras is not itself a protein kinase, it participates in the activation of Raf and other kinases, most likely through a phosphorylation mechanism. Once activated, Raf and other kinases phosphorylate MEK on two closely adjacent serine residues, S²¹⁸ and S²²² in the case of MEK-1, which are the prerequisite for activation of MEK as a kinase. MEK in turn phosphorylates MAP kinase on both a tyrosine, Y¹⁸⁵, and a threonine residue, T¹⁸³, separated by a single amino acid. This double phosphorylation activates MAP kinase at least 100-fold. Activated MAP kinase can then catalyze the phosphorylation of a large number of proteins, including several transcription factors and other kinaes. Many of these MAP kinase phosphorylations are mitogenically activating for the target protein, such as a kinase, a transcription factor, or another cellular protein. In addition to Raf-1 and MEKK, other kinases activate MEK, and MEK itself appears to be a signal integrating kinase. Current understanding is that MEK is highly specific for the phosphorylation of MAP kinase. In fact, no substrate for MEK other than the MAP kinase, ERK, has been demonstrated to date and MEK does not phosphorylate peptides based on the MAP kinase phosphorylation sequence, or even phosphorylate denatured MAP kinase. MEK also appears to associate strongly with MAP kinase prior to phosphorylating it, suggesting that phosphorylation of MAP kinase by MEK may require a prior strong interaction between the two proteins. Both this requirement and the unusual specificity of MEK are suggestive that it may have enough difference in its mechanism of action to other protein kinases that selective inhibitors of MEK, possibly operating through allosteric mechanisms rather than through the usual blockade of the ATP binding site, may be found.

It has been found that the compounds of the present invention are inhibitors of MEK and are useful in the treatment of a variety of proliferative disease states, such as conditions related to the hyperactivity of MEK, as well as diseases modulated by the MEK cascade.

### SUMMARY OF THE INVENTION

The present invention provides a compound of formula 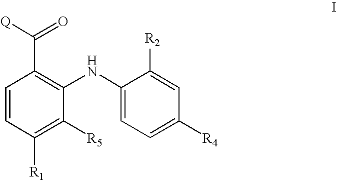
wherein
Q is C₁₋₆ alkyl, C₂₋₆ alkenyl, C₂₋₆ alkynyl, or C₂₋₅ heteroaryl, wherein the C₁₋₆ alkyl, C₂₋₆ alkenyl, and C₂₋₆ alkynyl groups are optionally substituted with between 1 and 2 hydroxy substituents, and the C₂₋₅ heteroaryl is optionally substituted with C₁₋₄ alkyl, and further wherein the C₁₋₄ alkyl is optionally substituted with between 1 and 3 substituents independently selected from hydroxy and amino;
R₁ and R₅ are each independently hydrogen or fluorine;
R₂ is hydrogen, chlorine, fluorine or methyl;
R₄ is bromine, chlorine, fluorine, iodine, C₁₋₆ alkyl, C₂₋₆ alkenyl, C₂₋₆ alkynyl, C₃₋₆ cycloalkyl, —(CH₂)—C₃₋₆ cycloalkyl, cyano, —O—(C₁₋₄ alkyl), —S—(C₁₋₂ alkyl), —SOCH₃, —SO₂CH₃, —SO₂NR₆R₇, —C≡C—(CH₂)ₙNH₂, —C≡C—(CH₂)ₙNHCH₃, —C≡C—(CH₂)ₙN(CH₃)₂, —C≡C—CH₂OCH₃, —C≡C(CH₂)ₙOH, —C≡C—(CH₂)ₙNH₂, (Z)-CHCHCH₂OCH₃, -(Z)-CHCH—(CH₂)ₙNHCH₃, (Z)-CHCH—(CH₂)ₙN(CH₃)₂, —(CH₂)ₚCO₂R₆, C(O)C₁₋₃ alkyl, C(O)NHCH₃, —(CH₂)ₘNH₂, —(CH₂)ₘNHCH₃, —(CH₂)ₘN(CH₃)₂, —(CH₂)ₘOR₆, —CH₂S(CH₂)ₜ(CH₃), —(CH₂)ₚCF₃, —C≡CCF₃, —CH═CHCF₃, —CH₂CHCF₂, —CH═CF₂, —(CF₂)ᵥCF₃, —CH₂(CF₂)ₙCF₃, —(CH₂)ₜCF(CF₃)₂, —CH(CF₃)₂, —CF₂CF(CF₃)₂, or —C(CF₃)₃, wherein the C₁₋₆ alkyl and C₂₋₆ alkynyl are optionally substituted with between 1 and 3 substituents independently selected from hydroxy and alkyl;
R₆ and R₇ are each independently hydrogen, methyl, or ethyl;
m is 1 to 4;
n is 1 to 2;
p is 0 to 2;
t is 0 to 1;
v is 1 to 5;
and pharmaceutically acceptable salts, C₁₋₆ amides and C₁₋₆ esters thereof.

An embodiment of the present invention provide a compound of formula I, as defined above, and pharmaceutically acceptable salts thereof.

Additionally provided by the present invention are compounds of Formula I, wherein:
Q is C₁₋₆ alkyl, C₂₋₆ alkenyl, or C₂₋₅ heteroaryl, wherein the C₁₋₆ alkyl group is optionally substituted with between 1 and 2 hydroxy substituents and the C₂₋₅ heteroaryl is optionally substituted with methyl;
Q is C₁₋₄ alkyl optionally substituted with between 1 and 2 hydroxy substituents;
Q is C₂₋₅ heteroaryl containing from one to two heteroatoms independently selected from the group consisting of nitrogen, oxygen and sulfur, and further wherein the C₂₋₅ heteroaryl is optionally substituted with C₁₋₄ alkyl, and further wherein the C₁₋₄ alkyl is optionally substituted with between 1 and 3 substituents independently selected from hydroxy and amino; or
Q is methyl, —CH₂OH, —(CH₂)₄OH, butenyl, methyl-substituted oxazolyl, thiazolyl, methyl-substituted imidazolyl, or pyridinyl.

The present invention also provides compounds of Formula I wherein R₁ is fluorine; R₂ is fluorine; R₅ is fluorine; or R₁ and R₅ are fluorine.

Additionally, the present invention provides compounds of Formula I, wherein R₄ is:
iodine, C₁₋₆ alkyl, C₂₋₆ alkenyl, or C₂₋₆ alkynyl, and further wherein the C₁₋₆ alkyl, C₂₋₆ alkenyl, and C₂₋₆ alkynyl are optionally substituted with hydroxy;
iodine, C₁₋₄ alkyl, C₂₋₃ alkenyl, C₂₋₃ alkynyl, or S—CH₃, and further wherein the C₁₋₄ alkyl, C₂₋₃ alkenyl, and C₂₋₃ alkynyl are optionally substituted with hydroxy;
iodine;
C₁₋₄ alkyl, C₂₋₃ alkenyl, or C₂₋₃ alkynyl, and further wherein the C₁₋₄ alkyl, C₂₋₃ alkenyl, and C₂₋₃ alkynyl are optionally substituted with hydroxy; or
iodine, ethyl, —CH₂OH, ethenyl, or acetyl.

The invention also provides a pharmaceutical composition comprising a compound of Formula I and a pharmaceutically acceptable carrier.

Additionally, the invention provides a method of treating a proliferative disease in a patient in need thereof comprising administering a therapeutically effective amount of a compound of Formula I.

Furthermore, the invention provides methods of treating cancer, restenosis, psoriasis, autoimmune disease, atherosclerosis, osteoarthritis, rheumatoid arthritis, heart failure, chronic pain, and neuropathic pain in a patient in need thereof comprising administering a therapeutically effective amount of a compound of Formula I.

In addition, the invention provides a method for treating cancer in a patient in need thereof comprising administering a therapeutically effective amount of a compound of Formula I in combination with radiation therapy or at least one chemotherapeutic agent.

The invention also provides the use of a compound of Formula I for the manufacture of a medicament for the treatment of the disease states or diseases provided above.

### DETAILED DESCRIPTION OF THE INVENTION

Certain terms are defined below and by their usage throughout this disclosure.

The terms “halogen” or “halo” in the present invention refer to a fluorine, bromine, chlorine, and iodine atom or fluoro, bromo, chloro, and iodo. The terms fluorine and fluoro, for example, are understood to be equivalent herein.

Alkyl groups, such as “C₁₋₆ alkyl”, include aliphatic chains (i.e., hydrocarbyl or hydrocarbon radical structures containing hydrogen and carbon atoms) with a free valence. Alkyl groups are understood to include straight chain and branched structures. Examples include methyl, ethyl, propyl, isopropyl, butyl, isobutyl, sec-butyl, t-butyl, pentyl, 2-pentyl, 3-pentyl, isopentyl, neopentyl, (R)-2-methylbutyl, (S)-2-methylbutyl, 3-methylbutyl, 2,3-dimethylpropyl, hexyl, and the like. The term “C₁₋₆ alkyl” includes within its definition the terms “C₁₋₄ alkyl” and “C₁₋₂ alkyl”.

Alkenyl groups, such as C₂₋₆ alkenyl, are analogous to alkyl groups, but have at least one double bond (two adjacent sp2 carbon atoms). Depending on the placement of a double bond and substituents, if any, the geometry of the double bond may be entgegen (E), or zusammen (Z), cis, or trans. Similarly, alkynyl groups, such as C₂₋₆ alkynyl, have at least one triple bond (two adjacent sp carbon atoms). Unsaturated alkenyl or alkynyl groups may have one or more double or triple bonds, respectively, or a mixture thereof. Like alkyl groups, unsaturated groups may be straight chain or branched. Examples of alkenyls and alkynyls include vinyl, allyl, 2-methyl-2-propenyl, cis-2-butenyl, trans-2-butenyl, and acetyl. The term “C₂₋₆ alkenyl” includes within its definition the term “C₂₋₃ alkenyl” and the term “C₂₋₆ alkynyl” includes within its definition the term “C₂₋₃ alkynyl”.

Cycloalkyl groups, such as C₃₋₆ cycloalkyl, refer to a saturated hydrocarbon ring structure containing from 3 to 6 atoms. Typical C₃₋₆ cycloalkyl groups include cyclopropyl, cyclobutyl, cyclopentyl, cyclohexyl, and the like.

The term “heteroaryl” or “C₂₋₅ heteroaryl” as used herein, unless otherwise indicated, includes monocyclic aromatic heterocycles containing five or six ring members, of which from 1 to 4 can be heteroatoms selected, independently, from N, S and O, and bicyclic aromatic heterocycles containing from eight to twelve ring members, of which from 1 to 4 can be heteroatoms selected, independently, from N, S and O.

Heterocyclic radicals, which include but are not limited to heteroaryls, include: furyl, (is)oxazolyl, isoxazolyl, thiophenyl, thiazolyl, pyrrolyl, imidazolyl, 1,3,4-triazolyl, tetrazolyl, pyridinyl, pyrimidinyl, pyridazinyl, indolyl, and their nonaromatic counterparts. Further examples of heterocyclic radicals include thienyl, piperidyl, quinolyl, isothiazolyl, piperidinyl, morpholinyl, piperazinyl, tetrahydrofuryl, tetrahydropyrrolyl, pyrrolidinyl, octahydroindolyl, octahydrobenzothiofuranyl, octahydrobenzofuranyl, (iso)quinolinyl, naphthyridinyl, benzimidazolyl, and benzoxazolyl.

The present invention includes the hydrates and the pharmaceutically acceptable salts and solvates of the compounds defined by Formula I. The compounds of this invention can possess a sufficiently basic functional group, and accordingly react with any of a number of inorganic and organic acids, to form a pharmaceutically acceptable salt.

The term “pharmaceutically acceptable salt” as used herein, refers to salts of the compounds of Formula I which are substantially non-toxic to living organisms. Typical pharmaceutically acceptable salts include those salts prepared by reaction of the compounds of the present invention with a pharmaceutically acceptable mineral or organic acid. Such salts are also known as acid addition salts. Such salts include the pharmaceutically acceptable salts listed in Journal of Pharmaceutical Science, 1977;66:2-19, which are known to the skilled artisan.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Example of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, hydrobromide, iodide, acetate, propionate, decanoate, caprate, caprylate, acrylate, ascorbate, formate, hydrochloride, dihydrochloride, isobutyrate, caproate, heptanoate, propiolate, glucuronate, glutamate, propionate, phenylpropionate, salicylate, oxalate, malonate, succinate, suberate, sebacate, fumarate, malate, maleate, hydroxymateate, mandelate, mesylate, nicotinate, isonicotinate, cinnamate, hippurate, nitrate, stearate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydrozybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, hemi-tartrate, benzenesulfonate, methanesulfonate, ethanesulfonate, propanesulfonate, hydroxyethanesulfonate, 1-naphthalenesulfonate, 2-naphthalenesulfonate, 1,5-naphthalenedisulfonate, mandelate, tartarate, and the like. A preferred pharmaceutically acceptable salt is hydrochloride.

It should be recognized that the particular counterion forming a part of any salt of this inventions is usually not of a critical nature, so long as the salt as a whole is pharmacologically acceptable and as long as the counterion does not contribute undesired qualities to the salt as a whole. It is further understood that such salts may exist as a hydrate.

The enantiomers of compounds of the present invention can be resolved by one of ordinary skill in the art using standard techniques well-known in the art, such as those described by J. Jacques, et al., “Enantiomers, Racemates, and Resolutions”, John Wiley and Sons, Inc 1981. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The compounds of Formula I can be prepared by techniques and procedures readily available to one of ordinary skill in the art, for example by following the procedures as set forth in the following Schemes, or analogous variants thereof. These synthetic strategies are further exemplified in examples below. These schemes are not intended to limit the scope of the invention in any way.

As used herein, the following terms have the meanings indicated: “AcOH” refers to acetic acid; “CDI” refers to 1,1′-carbonyldiimidazole; Celite® refers to a filter agent which is acid washed and approximately 95% SiO₂; “CHCl₃” refers to chloroform; “CH₂Cl₂” and “DCM” refer to dichloromethane; “conc.” refers to concentrated; “DABCO” refers to 1,4-diazabicyclo[2.2.2]octane; “DIEA” refers to N,N-diisopropylethylamine; “DMA” refers to N,N-dimethylacetamide; “DMF” refers to N,N-dimethylformamide; “DMSO” refers to methyl sulfoxide; “DMT-MM” refers to 4-(4,6-dimethoxy-1,3,5-triazin-2-yl)-4-methylmorpholinium chloride; “EtOAc” refers to ethyl acetate; “EtOH” refers to ethanol; “Et₂O” refers to diethyl ether; “FMOC” refers to 9H-fluoren-9-ylmethyl ester; “h” refers to hours; “HCl” refers to hydrochloric acid; “Me” refers to methyl; “MeOH” refers to methanol; “Me₂SO₄” refers to dimethyl sulfate; “min” refers to minutes; “NaOH” refers to sodium hydroxide' “Na₂SO₄ refers to sodium sulfate; “N-MM” refers to N-methylmorpholine; “Pd/C” refers to palladium on carbon; “PE” refers to petroleum ether which can be substituted with hexanes; “(Ph₃P)₂PdCl₂” refers to dichlorobis (triphenylphosphine)palladium(II); “(Ph₃P)₄Pd” refers to tetrakis (triphenylphosphine) palladium (0); “PS” refers to polymer-supported; “R.T.” refers to room temperature; “sat” refers to saturated; “TEA” refers to triethylamine; “TFA” refers to trifluoroacetic acid; “THF” refers to “tetrahydrofuran; “TIPSCI” refers to 1,3-dichloro-1,1,3,3,-tetraisopropyl-disiloxane; “TLC” refers to thin layer chromatography and “TMS” refers to trimethylsilyl. All other terms and substituents, unless otherwise indicated, are previously defined.

All other terms and substituents, unless otherwise indicated, are previously defined. The reagents and starting materials are readily available to one of ordinary skill in the art. Schemes 1 and 2 provide syntheses of the compounds of Formula I.

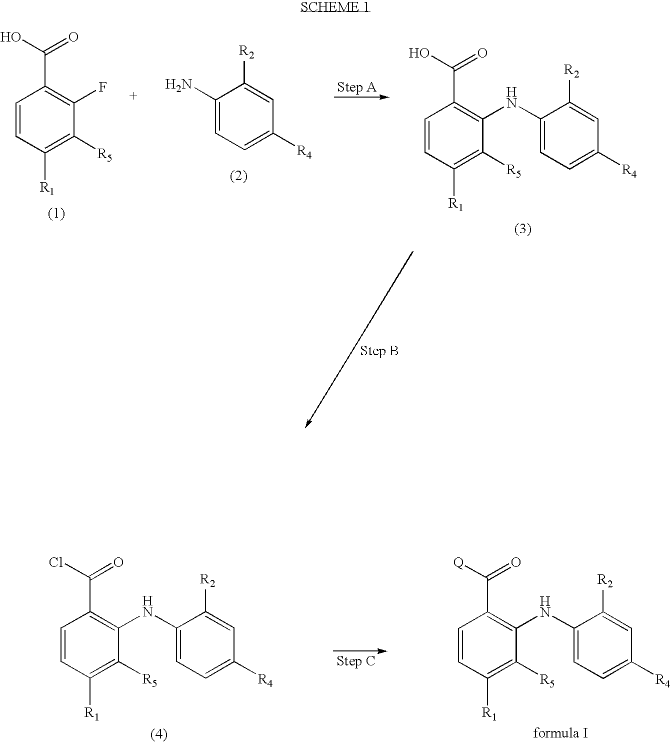

In Scheme 1, Step A, a suitable benzoic acid (1) is coupled with a suitable aniline (2) to provide a 2-(arylamino)-benzoic acid or diphenylamine (3). For example, the aniline (2) and the benzoic acid (1) are dissolved in a suitable organic solvent with an acid catalyst and heated at reflux for several hours. Preferred solvents are polar solvents such as ethanol, and preferred acid catalysts are mineral acids such as concentrated HCl. The reaction is typically complete within about 12 to 36 hours. The product (3) is typically isolated by filtration after cooling of the reaction mixture, and further purified, if desired, by standard methods such as chromatography or crystallization.

In Scheme 1, Step B, an acid chloride (4) is prepared from 2-(arylamino)-benzoic acid (3) by reacting the benzoic acid (3) with oxalyl chloride in a suitable solvent, such as DMF.

In Scheme 1, Step C, nucleophilic addition to an acid chloride (4), followed by deprotection and decarboxylation provides a compound of formula I. For example, an acid chloride (4) is combined with tris(trimethylsiloxy)ethylene and stirred under nitrogen. After the reaction is complete, dioxane and hydrochloric acid are added to the mixture and stirred to produce a compound of formula I. The product can be isolated by filtration and removing the solvent, for example by evaporation under reduced pressure, and further purified, if desired, by standard methods such as chromatography, crystallization, or distillation.

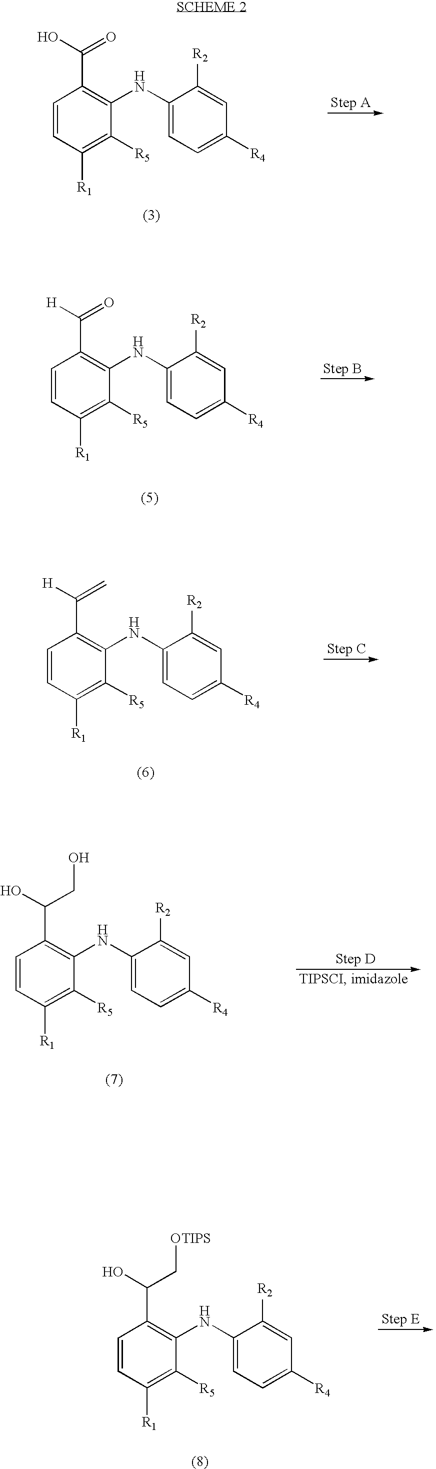

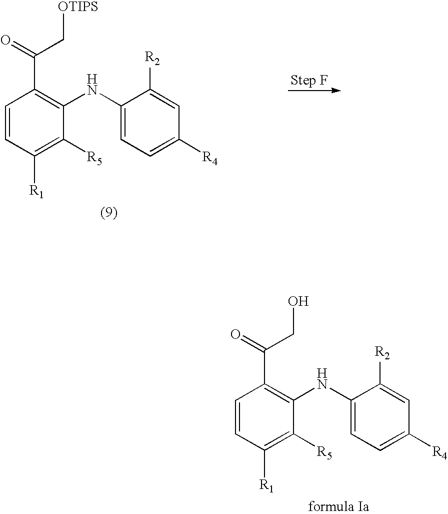

In Scheme 2, Step A, the benzoic acid (3) is converted to the benzaldehyde (5). For example, the benzoic acid (3) is reduced with lithium aluminum hydride in a suitable solvent, such as tetrahydrofuran providing an intermediate alcohol which is then oxidized with, for example, manganese dioxide in chloroform providing benzaldehyde (5). In Scheme 1, Steps B and C, the Wittig reaction, as known to one of skill in the art, is followed by dihydroxylation to provide compound (7). For example, the dihydroxylation is performed using a stoichiometric amount of 4-methyl-morpholine 4-oxide and a catalytic amount of OsO₄. In Step D, compound (7) is protected under procedures generally known in the art to provide the protected compound (8). In Step E, the protected compound (8) is oxidized according to procedures known in the art followed by a standard deprotection in Step F to provide a compound of formula Ia.

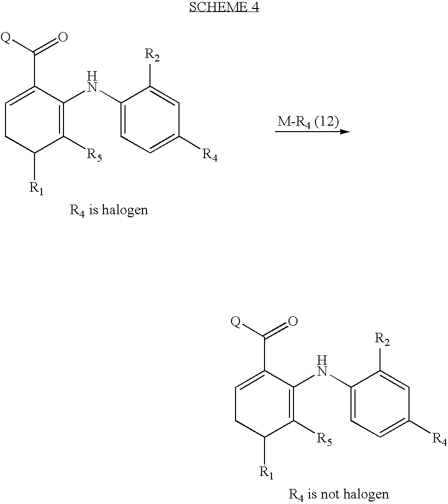

In Scheme 4, the compounds of formula 1, wherein R₄ is not halogen are prepared from the compounds of formula I wherein R₄ is halogen, by transition metal-promoted coupling with reagent M-R4 wherein R₄ is non-halogen (12) in a suitable solvent or solvents such as triethylamine, tetrahydrofuran or dimethylformamide. The transition metal-promoted coupling may be carried out with a palladium(0) or palladium (II) coupling agent, such as (Ph₃P)₄Pd or (Ph₃P)₂PdCl₂. The entire mixture is stirred from about 2 to 24 hours at room temperature. M is defined as a functional group known to transfer a carbon radical fragment in transition metal-promoted coupling processes. Examples of a suitable M group include trialkylstannyl, trialkylsilyl, trimethylsilyl, zinc, tin, copper, boron, magnesium and lithium. Examples of a suitable M-R4 reagent (12) when, R₄ is C₂₋₄ alkenyl is allyltributyltin or tetravinyltin, and when R₄ is hydroxy-substituted C₂₋₆ alkynyl is propargyl alcohol. Preferred halogens, when R₄ is halogen, are bromine and iodine.

The resulting compound of formula I, as well as the protected Formula I compound, can be isolated by removing the solvent, for example by evaporation under reduced pressure, and further purified, if desired, by standard methods such as chromatography, crystallization, or distillation.

It would be understood by one of skill in the art that the substituent R₄, when R₄ is non-halogen, may be further transformed, such as by oxidation, reduction, deprotection, or hydrogenation.

A compound wherein R₄ is C₂₋₄ alkenyl may be transformed to a compound wherein R₄ is hydroxy-substituted alkyl by treating the double bond of the alkene with ozone and NaBH₄. Furthermore, a compound wherein R₄ is C₂₋₄ alkenyl may be transformed to a compound wherein R₄ is alkyl substituted with 2 hydroxy substituents by treating the double bond of the alkene with OsO₄.

A compound wherein R₄ is an alkene or alkyne derivative may be reduced under conditions known in the art, such as through hydrogenation, such as with Pd/C under an atmosphere of hydrogen. For example, the alkyne derivative is dissolved in a suitable solvent, such as absolute ethanol, in the presence of a metal catalyst, such as palladium on carbon. This mixture is stirred under an atmosphere of hydrogen from about 1 to 24 hours at room temperature to provide the fully saturated derivative. Alternately, the alkyne derivative is partially reduced via hydrogenation to provide the alkene derivative. For example, the alkyne derivative is dissolved in a suitable solvent, such as tetrahydrofuran, in the presence of a catalyst, such as Lindlar catalyst or palladium on carbon and, if desired, a suitable compound which disrupts the actions of the catalyst, such as quinoline or pyridine. This mixture is stirred under an atmosphere of hydrogen from about 1 to 24 hours at room temperature to provide the alkene derivative.

The substituent R₄ may also be transformed into a different R₄ through standard synthetic procedures known to one of skill in the art.

It would be understood by one of skill in the art that the transformation of R₄ as shown in Scheme 4 may be performed at various steps throughout the synthesis of compounds of the present invention, as desired. For example, R₄ may be transformed before the coupling of the ester (1) and aniline (2) as shown in Scheme 1, Step A.

Further transformations of R4 are shown in Scheme 5 below.

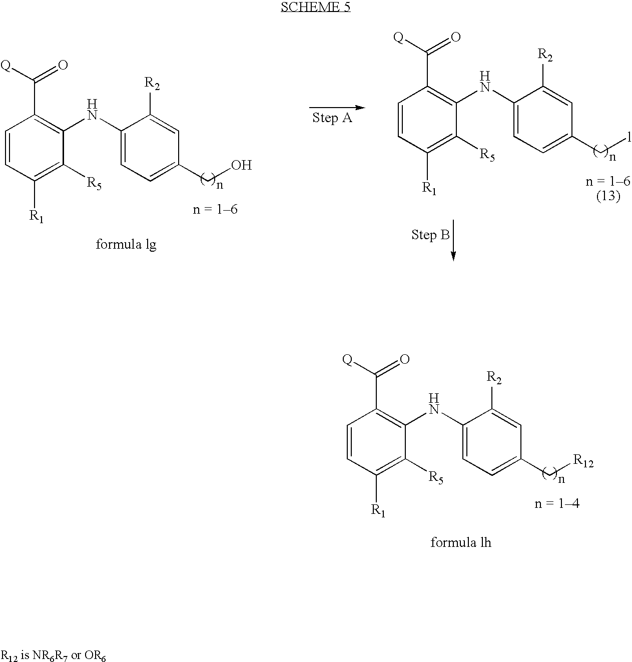

In Scheme 5, step A, the compound of formula Ig is dissolved in a suitable solvent such as tetrahydrofuran and reacted with methanesulfonyl chloride to give the intermediate mesylate, then Nal in EtOAc to give the iodide compound (13).

In Scheme 5, step B, the iodide compound (13) is reacted with a suitable amine, such as methylamine or dimethylamine, or a suitable alkoxide to give compounds of formula Ih.

It would also be understood by one of skill in the art that an aniline (2) may be prepared to include the desired R₄.

The aniline (2) can be prepared by techniques and procedures readily available to one of ordinary skill in the art and by following the procedures as set forth in the following Schemes, or analogous variants thereof. Additionally, anilines (2) are taught in U.S. Ser. No. 10/349,801 filed Jan. 23, 2003 and U.S. Ser. No. 10/349,826 filed Jan. 23, 2003, the disclosure of which is hereby incorporated by reference. These Schemes are not intended to limit the scope of the invention in any way.

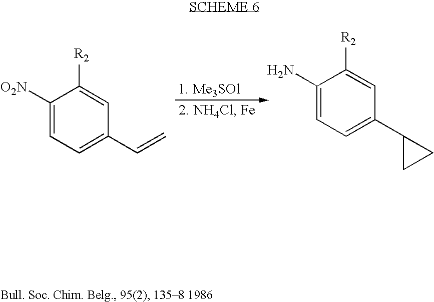

In Scheme 6, a suitably substituted para-nitrostyrene is reacted with dimethyloxosulfonium methylide to form the substituted para-nitrocyclopropylbenzene. Reduction of para-nitrocyclopropylbenzene with iron in the presence of weak acid gives the desired aniline.

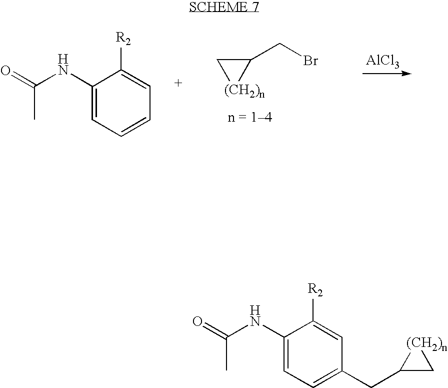

In Scheme 7, the suitable ortho-substituted acetamide is reacted with bromocyclobutane, bromocyclopropane, or bromocyclohexane under typical Friedel-Craft conditions, as known to one of skill in the art, to give the desired para-cycloalkylanilines. The acetamide is deprotected under conditions known to one of skill in the art to provide the desired para-cycloalkylmethylanilines.

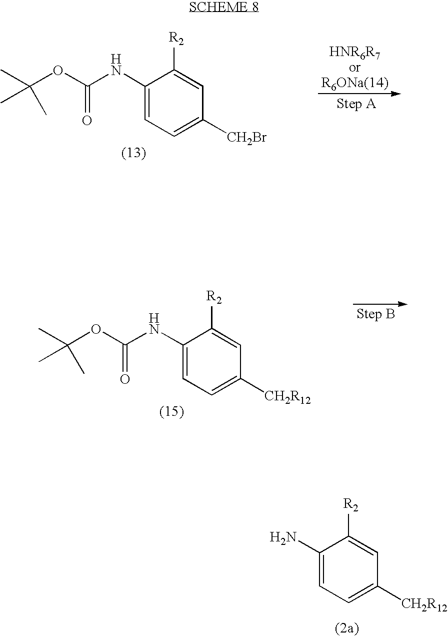

In Scheme 8, Step A, a suitable amine or alkoxide (14) is reacted with a 4-tert-butoxycarbonylamino-3-substituted-benzyl bromide (13), such as 4-tert-butoxycarbonylamino-3-fluorobenzyl bromide (J. Med. Chem., 2000;43:5017). In Step B, the BOC protecting group of compound of structure (15) is hydrolized with, for example, TFA, to provide the desired aniline (2a).

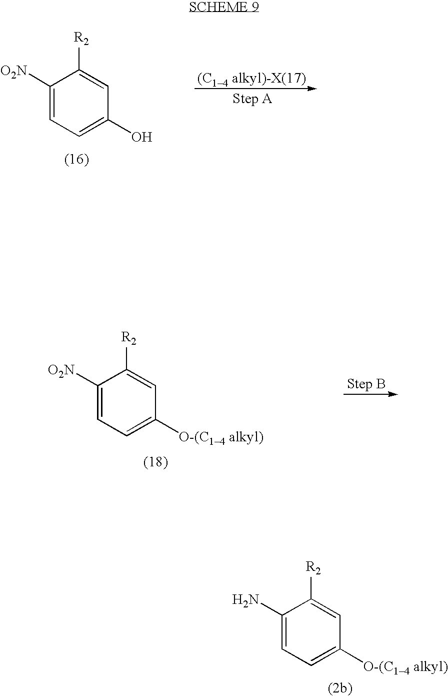

In Scheme 9, Step A, a suitable 3-substituted-4-nitrophenol (16), such as 3-fluoro-4-nitrophenol, is alkylated with a compound of structure (17) in the presence of a suitable base to provide a compound of structure (18). In Step B, compound (18) is reduced via hydrogenation in the presence of a metal catalyst, such as palladium on carbon, in an atmosphere of hydrogen to provide the desired aniline (2b).

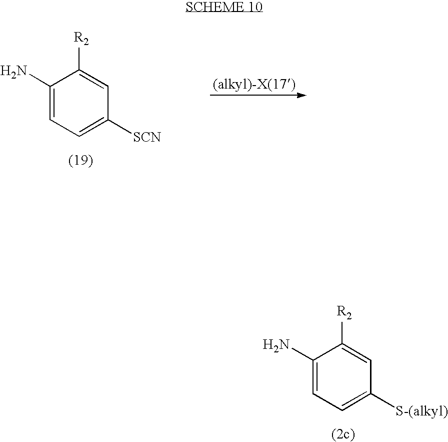

In Scheme 10, a suitable 4-(aminophenyl)thiocyanate (19), is alkylated with a compound of structure (17′) in the presence of a suitable nucleophilic base to provide an alkylthio compound of structure (2c). After reaction under standard conditions to form the diphenylamine (3), wherein R₄ is -S-(alkyl), as in Scheme 1 above, this compound is then oxidized to the corresponding sulfonyl compound, also generally, the diphenylamine (3), wherein R₄ is —SO₂-(alkyl).

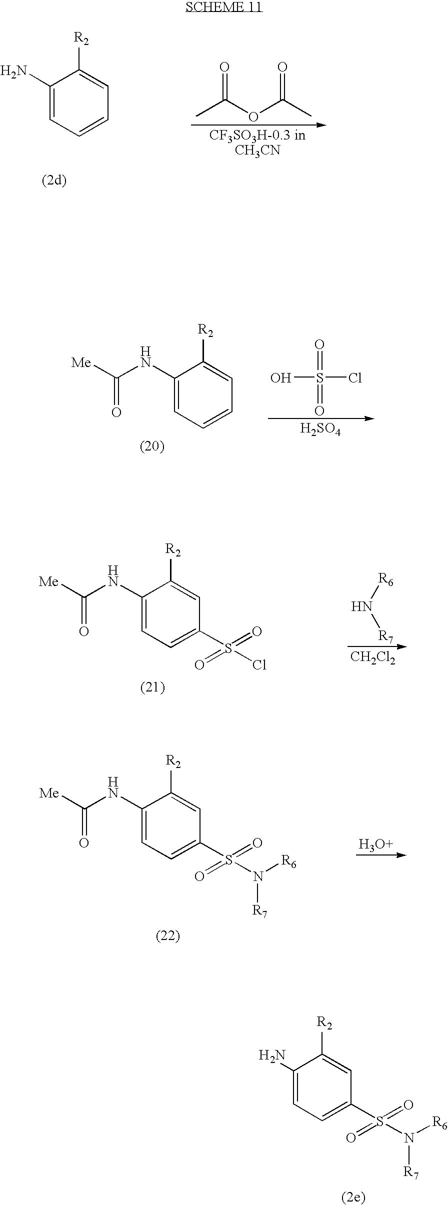

In Scheme 11, the proper ortho-substituted or unsubstituted aniline (2d) is acetylated with acetic anhydride in the presence of trifluoromethanesulfonic acid indium salt to give the protected aniline (20). Chlorosulfonation in the typical manner, as known in the art, gives the sulfonyl chloride derivative (21) which is reacted with an excess of a suitable amine in a solvent such as dichloromethane or dichloroethane to give the protected para-aminobenzenesulfonamide (22). Acid-mediated deprotection in the appropriate solvent gives the desired aniline (2e).

Alternatively, the desired aniline (2e) wherein R₂ is methyl, fluorine or chlorine, using compound (21) as the starting material can be prepared. Where R₂ is fluorine, the sulfonyl chloride derivative (21) is a compound known in the literature (German Patent DE 2630060, 1.978). Similarly, where R₂ is methyl, the sulfonyl chloride derivative (21) is also known in the literature (German Patent. DE 2750170, 1978). Finally, the sulfonyl chloride derivative (21) where R₂ is chlorine is commercially available.

In addition to the procedure described in Scheme 11, one of ordinary skill in the art would appreciate that there are numerous ways of acetylating anilines. For example, heating the aniline and acetic anhydride together in a suitable solvent, such as acetic acid, would achieve the same result.

Compounds of the present invention include, but are not limited to the following compounds:
1-[3,4-Difluoro-2-(2-fluoro-4-iodo-phenylamino)-phenyl]-2-hydroxy-ethanone;
[2-(4-Ethynyl-2-fluoro-phenylamino)-3,4-difluoro-phenyl]-pyridin-2-yl-methanone;
[2-(4-Ethynyl-2-fluoro-phenylamino)-3,4-difluoro-phenyl]-thiazol-2-yl-methanone;
[2-(4-Ethynyl-2-fluoro-phenylamino)-3,4-difluoro-phenyl]-( 1-methyl-1H-imidazol-2-yl)-methanone;
[3,4-Difluoro-2-(2-fluoro-4-iodo-phenylamino)-phenyl]-(4-methyl-oxazol-2-yl)-methanone;
1-[3,4-Difluoro-2-(2-fluoro-4-iodo-phenylamino)-phenyl]-ethanone;
1-{3,4-Difluoro-2-[2-fluoro-4-(2-hydroxy-ethyl)-phenylamino]-phenyl)-2-hydroxy-ethanone;
1-[2-(4-Ethyl-2-fluoro-phenylamino)-3,4-difluoro-phenyl]-2-hydroxy-ethanone;
1-[2-(4-Ethynyl-2-fluoro-phenylamino)-3,4-difluoro-phenyl]-ethanone;
1-[2-(4-Ethyl-2-fluoro-phenylamino)-3,4-difluoro-phenyl]-5-hydroxy-pentan-1-one;
1-[3,4-Difluoro-2-(2-fluoro-4-vinyl-phenylamino)-phenyl]-2-hydroxy-ethanone; and
1-[2-(4-Ethynyl-2-fluoro-phenylamino)-3,4-difluoro-phenyl]-pent-4-en-1-one.

As used herein, the term “patient” refers to any warm-blooded animal such as, but not limited to, a human, horse, dog, guinea pig, or mouse. Preferably, the patient is human.

The term “treating” for purposes of the present invention refers to treatment, prophylaxis or prevention, amelioration or elimination of a named condition once the condition has been established.

Selective MEK 1 or MEK 2 inhibitors are those compounds which inhibit the MEK 1 or MEK 2 enzymes, respectively, without substantially inhibiting other enzymes such as MKK3, PKC, Cdk2A, phosphorylase kinase, EGF, and PDGF receptor kinases, and C-src. In general, a selective MEK 1 or MEK 2 inhibitor has an IC₅₀ for MEK 1 or MEK 2 that is at least one-fiftieth (1/50) that of its IC₅₀ for one of the above-named other enzymes. Preferably, a selective inhibitor has an IC₅₀ that is at least 1/100, more preferably 1/500, and even more preferably 1/1000, 1/5000, or less than that of its IC₅₀ or one or more of the above-named enzymes.

The disclosed compositions are useful as both prophylactic and therapeutic treatments for diseases or conditions related to the hyperactivity of MEK, as well as diseases or conditions modulated by the MEK cascade. Examples include, but are not limited to, stroke, septic shock, heart failure, osteoarthritis, rheumatoid arthritis, organ transplant rejection, and a variety of tumors such as ovarian, lung, pancreatic, brain, prostatic, and colorectal.

The invention further relates to a method for treating proliferative diseases, such as cancer, restenosis, psoriasis, autoimmune disease, and atherosclerosis. Other aspects of the invention include methods for treating MEK-related (including ras-related) cancers, whether solid or hematopoietic. Examples of cancers include brain, breast, lung, such as non-small cell lung, ovarian, pancreatic, prostate, renal, colorectal, cervical, acute leukemia, and gastric cancer. Further aspects of the invention include methods for treating or reducing the symptoms of xenograft (cell(s), skin, limb, organ or bone marrow transplant) rejection, osteoarthritis, rheumatoid arthritis, cystic fibrosis, complications of diabetes (including diabetic retinopathy and diabetic nephropathy), hepatomegaly, cardiomegaly, stroke (such as acute focal ischemic stroke and global cerebral ischemia), heart failure, septic shock, asthma, Alzheimer's disease, and chronic or neuropathic pain. Compounds of the invention are also useful as antiviral agents for treating viral infections such as HIV, hepatitis (B) virus (HBV), human papilloma virus (HPV), cytomegalovirus (CMV), and Epstein-Barr virus (EBV). These methods include the step of administering to a patient in need of such treatment, or suffering from such a disease or condition, a therapeutically effective amount of a disclosed compound of formula I or pharmaceutical composition thereof.

The term “chronic pain” for purposes of the present invention includes, but is not limited to, neuropathic pain, idiopathic pain, and pain associated with chronic alcoholism, vitamin deficiency, uremia, or hypothyroidism. Chronic pain is associated with numerous conditions including, but not limited to, inflammation, arthritis, and post-operative pain.

As used herein, the term “neuropathic pain” is associated with numerous conditions which include, but are not limited to, inflammation, postoperative pain, phantom limb pain, burn pain, gout, trigeminal neuralgia, acute herpetic and postherpetic pain, causalgia, diabetic neuropathy, plexus avulsion, neuroma, vasculitis, viral infection, crush injury, constriction injury, tissue injury, limb amputation, arthritis pain, and nerve injury between the peripheral nervous system and the central nervous system.

The invention also features methods of combination therapy, such as a method for treating cancer, wherein the method further includes providing radiation therapy or chemotherapy, for example, with mitotic inhibitors such as a taxane or a vinca alkaloid. Examples of mitotic inhibitors include paclitaxel, docetaxel, vincristine, vinblastine, vinorelbine, and vinflunine. Other therapeutic combinations include a MEK inhibitor of the invention and an anticancer agent such as cisplatin, 5-fluorouracil or 5-fluoro-2-4(1H,3H)-pyrimidinedione (5FU), flutamide, and gemcitabine.

The chemotherapy or radiation therapy may be administered before, concurrently, or after the administration of a disclosed compound according to the needs of the patient.

Those skilled in the art will be able to determine, according to known methods, the appropriate therapeutically-effective amount or dosage of a compound of the present invention to administer to a patient, taking into account factors such as age, weight, general health, the compound administered, the route of administration, the type of pain or condition requiring treatment, and the presence of other medications. In general, an effective amount or a therapeutically-effective amount will be between about 0.1 and about 1000 mg/kg per day, preferably between about 1 and about 300 mg/kg body weight, and daily dosages will be between about 10 and about 5000 mg for an adult subject of normal weight. Commercially available capsules or other formulations (such as liquids and film-coated tablets) of 100, 200, 300, or 400 mg can be administered according to the disclosed methods.

The compounds of the present invention are preferably formulated prior to administration. Therefore, another aspect of the present invention is a pharmaceutical composition comprising a compound of Formula I and a pharmaceutically acceptable carrier. In making the compositions of the present invention, the active ingredient, such as a compound of Formula I, will usually be mixed with a carrier, or diluted by a carrier or enclosed within a carrier. Dosage unit forms or pharmaceutical compositions include tablets, capsules, pills, powders, granules, aqueous and nonaqueous oral solutions and suspensions, and parenteral solutions packaged in containers adapted for subdivision into individual doses.

Dosage unit forms can be adapted for various methods of administration, including controlled release formulations, such as subcutaneous implants. Administration methods include oral, rectal, parenteral (intravenous, intramuscular, subcutaneous), intracisternal, intravaginal, intraperitoneal, intravesical, local (drops, powders, ointments, gels, or cream), and by inhalation (a buccal or nasal spray).

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Carriers for solid dosage forms include (a) fillers or extenders, (b) binders, (c) humectants, (d) disintegrating agents, (e) solution retarders, (f) absorption acccelerators, (g) adsorbants, (h) lubricants, (i) buffering agents, and (j) propellants.

Compositions may also contain adjuvants such as preserving, wetting, emulsifying, and dispensing agents; antimicrobial agents such as parabens, chlorobutanol, phenol, and sorbic acid; isotonic agents such as a sugar or sodium chloride; absorption-prolonging agents such as aluminum monostearate and gelatin; and absorption-enhancing agents.

The following examples represent typical syntheses of the compounds of the present invention as described generally above. These examples are illustrative only and are not intended to limit the invention in any way. The reagents and starting materials are readily available to one of ordinary skill in the art.

### Preparation 1

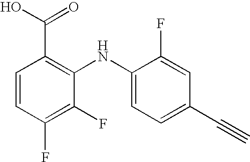

### 3,4-difluoro-2-[(4-ethynyl-2-fluorophenyl)amino]benzoic acid

### Step A: Preparation of 2-fluoro-4-[(trimethylsilyl)ethynyl]aniline

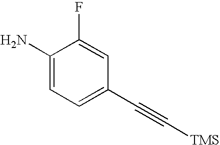

2-Fluoro-4-iodoaniline (5.00 g, 21.1 mmol), Cul (90 mg, 0.42 mmol), and (Ph₃P)₂PdCl₂ (300 mg, 0.42 mmol) were weighed into a flask which was sealed and flushed with N₂. A solution of TMS-acetylene (2.28 g, 23.2 mmol) in TEA (20 mL) was added, then the entire mixture stirred 15 hours at room temperature. The reaction mixture was diluted with diethyl ether (200 mL), filtered through Celite®, then all solvents removed under reduced pressure. The resulting dark brown oil was purified by filtration through a plug of flash silica (5% EtOAc/hexanes as eluant) to afford the desired product as a pale brown oil which rapidly solidified to give a crystalline solid (3.85 g, 88%); m.p. (EtOAc/hexanes) 45-47° C. ¹H NMR (400 MHz, CDCl₃) δ 7.10 (dd, J=11.7, 1.8 Hz, 1 H), 7.06 (ddd, J=8.3, 1.8, 1.0 Hz, 1 H), 6.66 (dd, J=9.4, 8.3 Hz, 1 H), 3.86 (br s, 2 H), 0.22 (s, 9 H). Anal. Calcd for C₁₁H₁₄FNSi: C, 63.7; H, 6.8; N, 6.8. Found: C, 63.7; H, 6.9; N, 6.7.

### Step B: Preparation of 3,4-difluoro-2-[[2-fluoro-4(trimethylsilylethynyl)phenyl]amino]benzoic acid

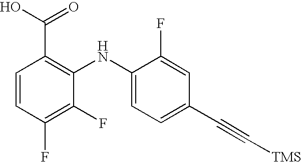

A mixture of the product of Step A, 2-fluoro-4-[(trimethylsilyl)ethynyl]aniline (3.85 g, 18.6 mmol) and 2,3,4-trifluorobenzoic acid (3.27 g, 18.6 mmol) was dissolved in dry THF (25 mL). The flask was fitted with a pressure-equalising dropping funnel and the entire apparatus evacuated and flushed with N₂. The solution was then cooled to −78° C. (acetone/dry ice) and a solution of 1.06 M LiHMDS (52.64 mL, 55.8 mmol) was added dropwise from the dropping funnel. Following this addition, the reaction mixture was allowed to warm to room temperature and stirred for a further 15 hours. The reaction solvent was removed under reduced pressure and the resulting residue partitioned between 1 M HCl (100 mL) and EtOAc (2×100 mL). The combined EtOAc fractions were then washed with water (100 mL) and saturated NaCl (100 mL), dried (Na₂SO₄), and the EtOAc removed under reduced pressure to afford a crude product which was purified by chromatography on flash silica (10% EtOAc/hexanes as eluant), giving the desired product as a pale yellow solid (3.99 g, 59%); m.p. (EtOAc/hexanes) 164-167° C. ¹H NMR [400 MHz, (CD3)2SO] δ 13.70 (br s, 1 H), 9.31 (br s, 1 H), 7.82 (ddd, J=9.1, 6.1, 2.0 Hz, 1 H), 7.34 (dd, J=12.0, 1.9 Hz, 1 H), 7.18 (ddd, J=8.3, 1.9, 0.8 Hz, 1 H), 7.16 (td, J=9.5, 7.3 Hz, 1 H), 6.93 (ddd, J=8.9, 8.3, 5.4 Hz, 1 H), 0.22 (s, 9 H). Anal. Calcd for C₁₈H₁₆F₃NO₂Si: C, 59.5; H, 4.4; N, 3.9. Found: C, 59.7; H, 4.7; N, 3.9.

### Step C: Preparation of 3,4-difluoro-2-[(4-ethynyl-2-fluorophenyl)amino]benzoic acid

The product of Step B, 3,4-difluoro-2-[[2-fluoro-4-(trimethylsilylethynyl) phenyl]amino]benzoic acid (3.99 g, 11.0 mmol), was dissolved in MeOH (200 mL), to which was added K₂CO₃ (3.03 g, 22.0 mmol). This mixture was stirred at room temperature for 15 hours, then the reaction solvent removed under reduced pressure. The resulting residue was dissolved in water (50 mL), to which was added 1 M HCl until the pH=4. The resulting pale brown precipitate was collected by filtration and dried to afford the desired product (3.17 g, 99%); m.p. (EtOAc/hexanes) 160-162° C. ¹H NMR [400 MHz, (CD3)2SO], δ 13.70 (br s, 1 H), 9.24 (br s, 1 H), 7.82 (ddd, J=9.2, 6.1, 2.1 Hz, 1 H), 7.38 (dd, J=12.0, 1.9, 1 H), 7.21 (ddd, J=8.4, 1.9, 0.8 Hz, 1 H), 7.16 (td, J=9.5, 7.3 Hz, 1 H), 6.96 (ddd, J=8.9, 8.4, 5.4 Hz, 1 H), 4.15 (s, 1 H). Anal. Calcd for C₁₅H₈F₃NO₂: C, 62.4; H, 3.1; N, 4.7. Found: C, 62.4; H, 3.2; N, 4.6.

### EXAMPLE 1

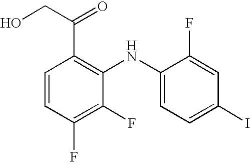

### 1-[3,4-Difluoro-2-(2-fluoro-4-iodo-phenylamino)-phenyl]-2-hydroxy-ethanone

To a stirring suspension comprised of 3,4-difluoro-2-(2-fluoro-4-iodo-phenylamino)-benzoic acid (1.33 g, 3.4 mmol, which can be prepared according to the procedure of WO 02/06213) and oxalyl chloride (1.4 mL, 16 mmoles) in dichloromethane (10 mL) at ambient temperature was added 0.025 mL of N,N-dimethylformamide. The reaction mixture was stirred for ten minutes and was concentrated in vacuo to the yellow solid 3,4-difluoro-2-(2-fluoro-4-iodo-phenylamino)-benzoyl chloride. The yellow solid was suspended in tris(trimethylsiloxy)ethylene (10 g, 33 mmol) and the stirring mixture was brought to 90° C. under a nitrogen atmosphere for six hours. The mixture was cooled slightly, and to it was added a solution consisting of dioxane (25 mL) and 10% aqueous hydrochloric acid (10 mL). Vigorous liberation of gas ensued and the mixture was stirred for ten minutes at 85° C. Brine (50 mL) was added and the mixture was extracted twice with ether (2×150 mL). The combined ether phases were washed twice with saturated aqueous sodium bicarbonate (2×150 mL). The ether phase was dried (MgSO4) and was concentrated in vacuo to 1.4 g of an orange oil that was purified by flash chromatography. Elution with a gradient (100% hexanes to 1:3 hexanes/ethyl acetate over 72 minutes) afforded a solid with minor impurities. The solid was dissolved in dichloromethane and vacuum filtered through silica to afford 0.135 g (9.8% yield over two steps) of a yellow solid; mp 128-128.5° C.; ¹H-NMR (400 MHz; CDCl₃) δ 9.80 (s, 1H), 7.40 (m, 3H), 6.77 (m, 2H), 4.83 (d, 2H, J=3.9 Hz), 3.44 (t, 1H, J=4.1 Hz); ¹⁹F-NMR (376 MHz; CDCl₃) δ −124.52 (t, 1F, J=10.1 Hz), −125.27 (dd, 1F, J=10.1, 5.1 Hz), −144.11 (d, 1F, J=7.7 Hz); MS (APCI+) 407.8 (M+1, 28), 389.8 (100); (APCI−) 405.8 (M−1, 100), 402.8 (53); IR 1662, 1529, 1503, 1260, 1067 cm⁻¹; % C(calculated for C₁₄H₉F₃INO₂/found) 41.30/41.52, % H 2.23/2.08, % N 3.44/3.45.

### EXAMPLE 2

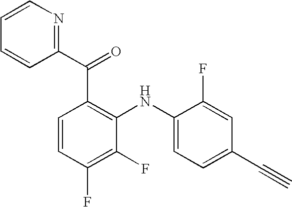

### [2-(4-Ethynyl-2-fluoro-phenylamino)-3,4-difluoro-phenyl]-pyridin-2-yl-methanone

n-Butyllithium (1.6 M in hexanes, 3.0 mL, 4.8 mmol) was added rapidly to a −78° C. solution of 2-bromopyridine (0.46 mL, 4.82 mmol) in tetrahydrofuran (5 mL). The resultant brown-colored reaction mixture was stirred 30 min at −78° C. A solution of the product of preparation 1, 2-(4-ethynyl-2-fluoro-phenylamino)-3,4-difluoro-benzoic acid (284 mg, 0.975 mmol), in tetrahydrofuran (5 mL) was added via cannula and the resultant brown-colored slurry was warmed to ambient temperature. After 1 h, the reaction was partitioned between water and ethyl acetate. The organics were washed with 1 M aqueous hydrochloric acid (2×2 mL), water, and brine, were dried over magnesium sulfate and concentrated in vacuo. Chromatography on silica gel (dichloromethane) afforded [2-(4-ethynyl-2-fluoro-phenylamino)-3,4-difluoro-phenyl]-pyridin-2-yl-methanone (158 mg, 46% yield) as a yellow film. Recrystallization from ether-hexanes afforded yellow needles: m.p. 109-111° C.; ¹H NMR (400 MHz, acetone-d₆) δ 8.77 (s, 1 H), 8.60 (ddd, J=4.9, 1.7, 1.0 Hz, 1 H), 8.04-7.96 (m, 2 H), 7.71 (ddd, J=8.9, 5.9, 2.1 Hz, 1 H), 7.59 (ddd, J=7.3, 4.6, 1.5 Hz, 1 H), 7.21-7.14 (m, 3 H), 6.95 (td, J=8.8, 4.9 Hz, 1 H), 3.59 (s, 1 H); ¹⁹F NMR (376 MHz, acetone-d₆) δ −132.2, −132.7, −145.1. Anal. Calcd/Found for C₂₀H₁₁F₃N₂O₁: C, 68.18/68.09; H, 3.15/3.13; 7.95/7.87.

### EXAMPLE 3

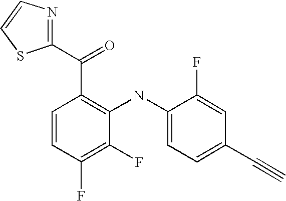

### [2-(4-Ethynyl-2-fluoro-phenylamino)-3,4-difluoro-phenyl]-thiazol-2-yl-methanone

### Step A: Preparation of 2-(4-Ethynyl-2-fluoro-phenylamino)-3,4-difluoro-N-methoxy-N-methyl-benzamide

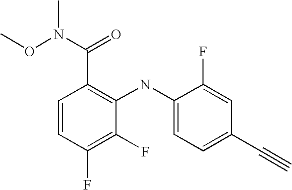

The product of of preparation 1, (2.2 g, 7.6 mmol) was dissolved in dichloromethane (30 mL) and N-methylmorpholine (3.5 mL). To the resultant solution was added N,O-dimethylhydroxylamine hydrochloride (0.95 g, 9.7 mmol) and PyBOP (3.9 g, 7.5 mmol). The resultant reaction mixture was stirred for 2 h at ambient temperature. An additional portion of PyBOP (1.3 g, 2.5 mmol) was added and stirring was continued. After 2 h, the reaction was diluted with ethyl acetate (100 mL) and washed with water (100 mL×3) and saturated brine (100 mL×3). The organics were dried over anhydrous sodium sulfate, concentrated in vacuo, and purified by silica gel chromatography to afford the product (1.33 g, 53% yield): ¹H NMR [400 MHz, (CD3)2SO] δ 8.03 (br s, 1 H), 7.29-7.22 (m, 3 H) 7.08 (dd, J=8.3, 1.5 Hz, 1 H), 6.67 (td, J=8.8, 2.0 Hz, 1 H), 4.05 (s, 1 H), 3.39 (s, 3 H), 3.07 (s, 3 H); MS (APCI+) for C₁₇H₁₃F₃N₂O₂=335.1 [M+1].

### Step B: Preparation of [2-(4-ethynyl-2-fluoro-phenylamino)-3,4-difluoro-phenyl]-thiazol-2-yl-methanone

A solution of thiazole (0.148 mL, 2.08 mmol) in tetrahydrofuran (10 mL) was cooled to −48° C. and treated with n-butyllithium (1.3 mL, 1.6 M in hexane, 2.08 mmol). The resultant purple-colored solution was stirred 15 min at −48° C. To this reaction mixture was added a solution of 2-(4-ethynyl-2-fluoro-phenylamino)-3,4-difluoro-N-methoxy-N-methyl-benzamide (200 mg, 0.6 mmol) in tetrahydrofuran (10 mL). The resultant deep yellow-colored reaction mixture was stirred an additional 30 min and was quenched with saturated aqueous ammonium chloride (30 mL). The reaction was partitioned with ethyl acetate and the organic layer was separated and was washed with saturated aqueous bicarbonate (50 mL×3). The organics were dried over anhydrous sodium sulfate, concentrated and chromatographed on silica gel. Recrystallization of the yellow solid from dichloromethane-hexanes afforded [2-(4-ethynyl-2-fluoro-phenylamino)-3,4-difluoro-phenyl]-thiazol-2-yl-methanone (88 mg, 41% yield): m.p. 161° C.; ¹H NMR (400 MHz, CDCl₃) □ 9.50 (s, 1 H), 8.60 (ddd, J=9.0, 5.9, 2.2 Hz, 1 H), 8.08 (d, J=3.2 Hz, 1 H), 7.74 (d, J=2.9 Hz, 1 H), 7.18 (m, 1 H), 6.91 (dt, J=7.1, 9.2 Hz, 1 H), 6.83 (dt, J=5.9, 8.3 Hz, 1 H), 3.03 (s, 1 H); ¹⁹F NMR (376 MHz, CDCl₃) δ −126.6, −129.2 −141.9. Anal. Calcd/Found for C₁₈H₉F₃N₂OS.0.12(C₄H₈O₂): C, 60.17/60.04; H, 2.72/3.11; N, 7.59/7.55.

### EXAMPLE 4

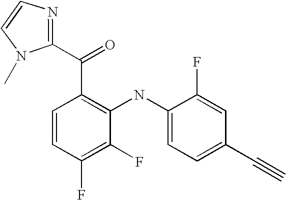

### [2-(4-Ethynyl-2-fluoro-phenylamino)-3,4-difluoro-phenyl]-(1-methyl-1H-imidazol-2-yl)-methanone

A solution of N-methyl imidazole (0.27 mL, 3.4 mmol) in tetrahydrofuran (10 mL) was cooled to −78° C. and treated with n-butyllithium (3.17 mL, 1.6 M in hexanes, 5.07 mmol). The resultant reaction mixture was stirred 15 min at −78° C. and 2 h at ambient temperature and was then transferred to a −78° C. solution of the product of preparation 1, 2-(4-ethynyl-2-fluoro-phenylamino)-3,4-difluoro-benzoic acid (200 mg, 0.69 mmol) in tetrahydrofuran (5 mL). The resultant reaction mixture was stirred 30 min at −78° C. and was allowed to warm to ambient temperature overnight. The reaction was partitioned between water (20 mL) and ethyl acetate (30 mL). The organic layer was washed with water (40 mL×3) and saturated brine (35 mL×2), dried over anhydrous sodium sulfate and concentrated in vacuo. Silica gel chromatography afforded a yellow solid. Recrystallization from dichloromethane-hexanes afforded [2-(4-Ethynyl-2-fluoro-phenylamino)-3,4-difluoro-phenyl]-(1-methyl-1H-imidazol-2-yl)-methanone (105 mg, 43% yield): m.p. 166° C.; ¹H NMR (400 MHz, CDCl₃) δ 9.08 (s, 1 H), 8.05 (ddd, J=9.0, 5.8, 2.2 Hz, 1 H), 7.22 (d, J=1.0 Hz, 1 H), 7.18-7.11 (m, 2 H), 7.11 (s, 1 H), 6.93 (dt, J=6.9, 9.0 Hz, 1 H), 6.73 (td, J=8.5, 6.3 Hz, 1 H), 4.03 (s, 3 H), 3.00 (s, 1 H); ¹⁹F NMR (376 MHz, CDCl₃) δ−128.8, −131.0, −141.4; MS (APCI+)=356.0. Anal. Calcd/Found for C₁₉H₁₂F₃N₃O.0.03(CH₂Cl₂): C, 63.87/63.76; H, 3.40/3.26; N, 11.74/11.69

### EXAMPLE 5

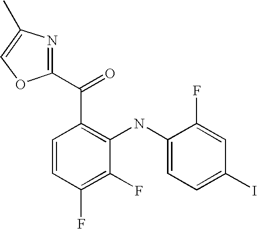

### [3,4-Difluoro-2-(2-fluoro-4-iodo-phenylamino)-phenyl]-(4-methyl-oxazol-2-yl)-methanone

### Step A: Synthesis of 3,4-Difluoro-2-(2-fluoro-4-iodo-phenylamino)-benzoyl chloride

A solution of 3,4-Difluoro-2-(2-fluoro-4-iodo-phenylamino)-benzoic acid (5.0 g, 12.7 mmol) in thionyl chloride (75 mL) containing 10 drops of dimethylformamide was heated at reflux for 15 min. The solvent was removed in vacuo affording 3,4-Difluoro-2-(2-fluoro-4-iodo-phenylamino)-benzoyl chloride (5.3 g, which can be prepared according to the procedure of WO 02/06213) as a yellow solid.

### Step B: Synthesis of [3,4-Difluoro-2-(2-fluoro-4-iodo-phenylamino)-phenyl]-(4-methyl-oxazol-2-yl)-methanone

A solution of 4-methyl-oxazole (0.080 mL, 1.0 mmol) in tetrahydrofuran (10 mL) was cooled to −78° C. and treated with n-butyllithium (1.6 M in hexane, 0.51 mL, 0.81 mmol). The resultant solution was stirred 25 min at −78° C. Zinc chloride (1.45 mL, 1.0 M in ether) was added and the reaction mixture was allowed to warm to 0° C. over 45 min. Copper (I) iodide (185 mg, 0.97 mmol) was added and the reaction was stirred for 10 min. 3,4-Difluoro-2-(2-fluoro-4-iodo-phenylamino)-benzoyl chloride (200 mg, 0.49 mmol) was added and the reaction mixture was allowed to warm to ambient temperature overnight. The reaction was diluted with ethyl acetate and was washed with concentrated ammonium hydroxide-water (1:1, 40 mL), water (40 mL) and brine (40 mL). The organics were concentrated in vacuo and chromatographed on silica gel. Elution with hexanes ethyl acetate (gradient to 20% ethyl acetate) afforded [3,4-Difluoro-2-(2-fluoro-4-iodo-phenylamino)-phenyl]-(4-methyl-oxazol-2-yl)-methanone (21 mg) after crystallization from dichlormethane-hexanes: m.p. 127° C.; ¹H NMR (400 MHz, CDCl₃) δ 9.59 (s, 1 H), 8.56 (ddd, J=9.3, 5.8, 2.1 Hz, 1 H), 7.62 (d, J=1.2 Hz, 1 H), 7.40 (dd, J=10.3, 2.0 Hz, 1 H), 7.34 (br d, J=8.6 Hz, 1 H), 6.86 (dt, J=7.1, 9.2 Hz, 1 H), 6.69 (dt, J=4.9, 8.7 Hz, 1 H), 2.30 (d, J=1.2 Hz, 3 H); ¹⁹F NMR (376 MHz, CDCl₃) δ −125.7, −125.9, −143.2; MS (APCI+)=459.0. Anal. Calcd/Found for C₁₇H₁₀F₃IN₂O₂.0.1(C₆H₁₄): C, 45.29/45.38; H, 2.46/2.32; N, 6.00/5.73.

### EXAMPLE 6

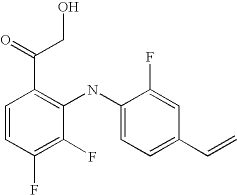

### 1-[3,4-difluoro-2-(2-fluoro-4-vinyl-phenylamino)-phenyl]-2-hydroxy-ethanone

### Step A: Synthesis of [3,4-Difluoro-2-(2-fluoro-4-iodod-phenylamino)-phenyl]-methanol

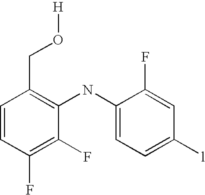

To 3,4-difluoro-2-(2-fluoro-4-iodo-phenylamino)-benzoic acid (10.0 g, 25.4 mmol) in tetrahydrofuran was added borane in tetrahydrofuran (38.2 mL of a 1.0M solution) after 16 hours stirring at room temperature, the reaction was complete and quenched by careful addition of 2% HCl (100 mL). The mixture was stirred for 1 h, brine was added and the organic layer was separated which was dried and evaporated to give a colorless solid (7.13 g 74%) that was recrystalized from hot dichloromethane/hexane to give an analytical sample. MS (APCI+)380 (M+1+); ¹H NMR(400 mHz, DMSO) 7.62(s,1H), 7.50(d,1H), 7.24(m,3H), 6.29(d of t, 1H), 5.35(t,1 H), 4.42(d,1H).

### Step B: Synthesis of 3,4-Difluoro-2-(2-fluoro-4-iodod-phenylamino) benzaldehyde

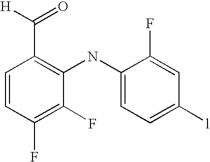

To [3,4-Difluoro-2-(2-fluoro-4-iodod-phenylamino)-phenyl]-methanol (17.1 g, 45 mmol) in dichloromethane was added manganese dioxide (8 equiv., 31 g) and the reaction was heated to reflux for 16 h. after cooling the reaction was filtered and the filtrate evaporated to give a yellow solid (14.15 g, 83%). MS (APCI−) 376 (M−1−); ¹H NMR(400 mHz, DMSO) 9.98(s,1H), 9.22(s,1 H), 7.73(ddd, 1 H), 7.61 (dd,1 H), 7.42(dt, 1 H), 7.22(m,1H), 6.85(dt,1H).

### Step C: Synthesis of (2,3-Difluoro-6-vinyl-phenyl)-(2-fluoro-4-iodo-phenyl)-amine

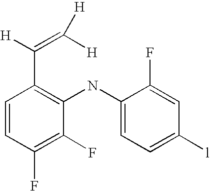

To methyl triphenylphosphonium bromide(13.6 g 2.2 equiv) in tetrahydrofuran(180 mL) at 0° C. was added n-butyllithium (2.2 equiv, 41 mL of 1.6M). The reaction was stirred 10 minutes giving a clear orange solution. 3,4-Difluoro-2-(2-fluoro-4-iodod-phenylamino) benzaldehyde(7.0 g, 18.6 mmol) was added giving a red solution. After 2 hours the reaction was poured into aqueous ammonium chloride solution. The reaction was extracted with ethyl acetate, washed with brine to give an oil after evaporation of the solvent. This oil was triturated with diethyl ether to remove much of the phosphine oxide by product. The crude material was used with out futher purification.

### Step D: Synthesis of 1-{3,4-difluoro-2-(2-fluoro-4-iodo-phenylamino)-phenyl]-ethane-1,2-diol

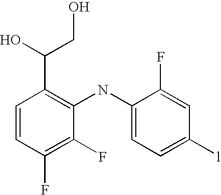

To crude (2,3-Difluoro-6-vinyl-phenyl)-(2fluoro-4-iodo-phenyl)-amine (˜4 mmol) in acetone water (8:1, 40 mL) was added N-methylmorpholine N-oxide(936 mg, 2 equiv.) and osmium tetraoxide (5 mol % in tert-butanol). The reaction was stirred at room temperature for 1.5 h and the solvent was evaporated and loaded directly onto a flash column. Chromatography with silica gel eluting with 3/2 diethylether/hexane gave product as a colorless oil that solidified on standing (1.53 g est 93%). MS (APCI+) 410 (M+1+); ¹H NMR(400 mHz, CDCl3), 7.39(dd,1H), 7.28(m,1H), 7.14(dt,1H), 7.00(dt,1H), 6.57(s, 1H), 6.33(dt,1 H), 4.90(m,1 H), 4.72(t,1 H), 2.91 (d,1 H), 1.95(t,1 H).

### Step E: Synthesis of 1-[3,4-difluoro-2-(2-fluoro-4-iodo-phenylamino)-phenyl]-2-triisopropylsilanyloxy-ethanol

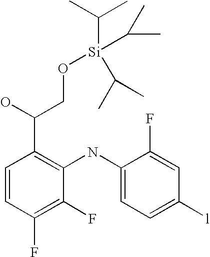

To 1-{3,4-difluoro-2-(2-fluoro-4-iodo-phenylamino)-phenyl]-ethane-1,2-diol (409 mg, 1.0 mmol)in dry dimethylformamide (3 mL) was added imidazole(136 mg, 2.0 mmol) and then triisopropylsilyl chloride. The reaction was stirred at room temperature for 3 h and then poured into water, the reaction was extracted with diethyl ether, the organic layer was washed with water and then brine. After drying over magnesium sulfate the solvent was removed under reduced pressure to provide product as a colorless oil(600 mg). MS (APCI+) 566 (M+1+); ¹H NMR(400 mHz, CDCl3) 7.37(dd,1H), 7.26(m,1H), 7.15(m,1H), 6.66(s,1H), 6.32(dt, 1H), 4.87(m,1H), 3.70(m,2H), 3.04(d,1H), 1.05-0.90(m, 21H).

### Step F: Synthesis of 1-[3,4-difluoro-2-(2-fluoro-4-iodo-phenylamino)-phenyl]-2-triisopropylsilanyloxy-ethanone.

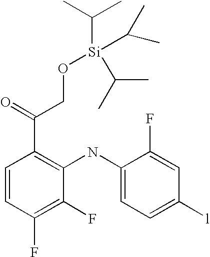

To 1-[3,4-difluoro-2-(2-fluoro-4-iodo-phenylamino)-phenyl]-2-triisopropylsilanyloxy-ethanol ( 300 mg, 0.50 mmol) in dichloromethane(10 mL), was added pyridinium dichromate (282 mg, 1.5 equiv). The reaction was heated to reflux for 4 h, then cooled and loaded directly onto a flash column. Chromatography on silica gel eluting with 2:1 hexane/dichloromethane provided product as a yellow oil(122 mg, 40%). MS (APCI+) 564 (M+1+); ¹H NMR(400 mHz, CDCl3) 9.75(s,1H), 7,67(ddd,1H), 7.40(dd,1H), 7.35(m,1H), 6.66(m,1H), 6.58(m, 1H), 4.83(s,2H), 1.10-1.00(m,21H).

### Step G: Synthesis of 1-[3,4-difluoro-2-(2-fluoro-4-vinyl-phenylamino)-phenyl]-2-hydroxy-ethanone

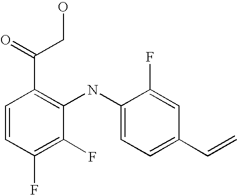

To 1-[3,4-difluoro-2-(2-fluoro-4-iodo-phenylamino)-phenyl]-2-triisopropylsilanyloxy-ethanone in dimethoxyethane/water ( 2:1, 3mL)was added tetrakistriphenylphosphine Pd (5%, 18 mg). This solution was stirred for 20 min, potassium carbonate and 2,4,6-trivinyl-cyclotriboroxane pyridine complex (77 mg, 1 equiv) was added. The reaction was heated at reflux for 3 h, thin layer analysis shows that the reaction was complete. The reaction was cooled and tetrabutyl ammonium fluoride (0.45 mL of 1.0M solution, 1.5 equiv.) and (10% HCl, 2 mL) was added. The mixture was heated at reflux for 4 h, then cooled and poured into ethyl acetate, washed with water and then saturated sodium bicarbonate to give a brown oil. Chromatography of the crude product on silica gel eluting with 1:4 hexane/dichloromethane gave product as a yellow solid (34 mg, 35%). MS (APCI+) 308 (M+1+); ¹H NMR(400 mHz, CDCl3) 9.90(s,1H), 7.43(m,1H), 7.18(dd, 1H), 7.10(d,1H), 6.98(m,1H), 6.75(m, 1H), 6.65(dd,1 H), 5.65(d,1 H), 5.23(d,1 H), 4.83(d,2H), 3.44(t,1H).

### EXAMPLE 7

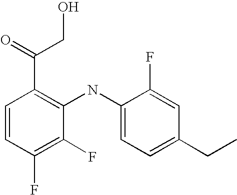

### 1-[2-(4-ethyl-2-fluoro-phenylamino)-3,4-difluoro-phenyl]-2-hydroxy-ethanone

### Step A: Synthesis of 1-[3,4-difluoro-2-(2-fluoro-4-vinyl-phenylamino)-phenyl]-2-triisopropylsilanyloxy-ethanol.

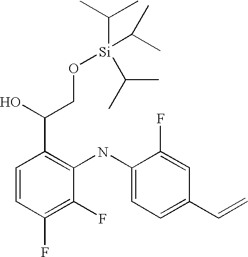

To 1-[3,4-difluoro-2-(2-fluoro-4-iodo-phenylamino)-phenyl]-2-triisopropylsilanyloxy-ethanol from Example 6 step E (1.94 g, 3.4 mmol) using the palladium coupling procedure of Exampe 6 step G provided product after silica gel chromatography 1:1 hexane/diethyl ether as a colorless oil (1.13 g, 72%). MS (APCI+) 466 (M+1+).

### Step B: Synthesis of 1-[2-(4-ethyl-2-fluoro-phenylamino)-3,4-difluoro-phenyl]-2-triisopropylsilanyloxy-ethanol

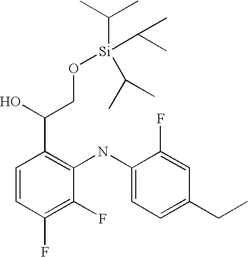

1-[3,4-Difluoro-2-(2-fluoro-4-vinyl-phenylamino)-phenyl]-2-triisopropylsilanyloxy-ethanol (500 mg, 1.07 mmol) was reduced employing 5% Pt on C in tetrahydrofuran at 55 psi hydrogen for 2 h. The reaction was judged complete by hydrogen uptake, the catalyst was removed by filtration and the product was isolated by evaporation of the solvent. The product (445 mg, 89%) was employed directly in the next reaction. MS (APCI+) 468 (M+1+).

### Step C: Synthesis of 1-[2-(4-ethyl-2-fluoro-phenylamino)-3,4-difluoro-phenyl]-2-triisopropylsilanyloxy-ethanone

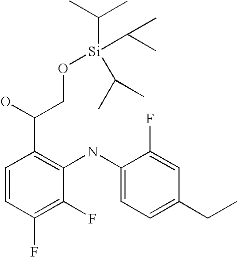

1-[2-(4-Ethyl-2-fluoro-phenylamino)-3,4-difluoro-phenyl]-2-triisopropylsilanyloxy-ethanol (445 mg, 0.95 mmol) was oxidized as in Example 6 step F, to provide product as a yellow oil(270 mg, 60%). MS (APCI+) 466 (M+1+); ¹H NMR(400 mHz, CDCl3) 9.87(s,1H), 7.64(dt, 1H), 6.8-7.0(m,3H), 6.64(m,1H), 4.86(s,2H), 2.61 (q,2H), 1.22(t,3H), 1.2-1.0(m,21H).

### Step D: Synthesis of 1-[2-(4-ethyl-2-fluoro-phenylamino)-3,4-difluoro-phenyl]-2-hydroxy-ethanone

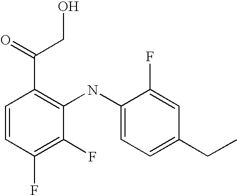

To 1-[2-(4-ethyl-2-fluoro-phenylamino)-3,4-difluoro-phenyl]-2-triisopropylsilanyloxy-ethanone (270 mg) in tetrahydrofuran (10 mL) was added 10% HCl (1.5 mL). the reaction was heated at reflux for 3 h and then poured into ethylacetate, washed with water and then saturated sodium bicarbonate to provide a yellow oil. Chromatography of the crude product in silica gel (1:4 hexane/dichloromethane provided product (48 mg, 27%). MS (APCI+) 310 (M+1+); ¹H NMR(400 mHz, CDCl3) 9.93(s,1H), 7.42(m,1H), 7.00(dt,1H), 6.92(m,2H), 6.66(m,1H), 4.83(s,2H), 3.52(br s,1H), 2.63(q,2H), 1.22(t,3H).

### EXAMPLE8

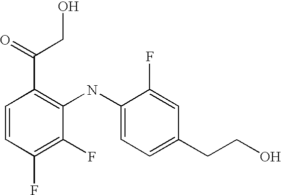

### 1-{3,4-difluoro-2-[2-fluoro-4-(2-hydroxy-ethyl)-phenylamino]-phenyl}-2-hydroxyl-ethanone

### Step A: Synthesis of 1-{3,4-difluoro-2-[2-fluoro-4-(2-hydroxy-ethyl)-phenylamino]-phenyl}-2-triisopropylsilanyloxy-ethanol

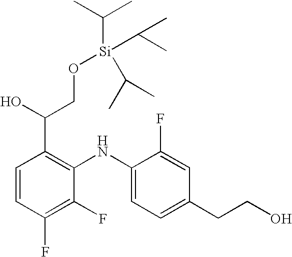

To 1-[3,4-difluoro-2-(2-fluoro-4-vinyl-phenylamino)-phenyl]-2-triisopropylsilanyloxy-ethanol (634 mg, 1.35 mmol) in dry tetrahydrofuran (15 mL) at 0° C. was added borane in tetrahydrofuran (2.7 mL, of 1.0M solution). After 1.5 h sodium perborate hydrate (805 mg, 6 equiv.) in water (15 mL) was added. The reaction was stirred for 3 hours, poured into water and extracted with ethyl acetate to give a colorless oil that was used without further purification. MS (APCI−) 310 (M−1−) 482.

### Step B: Synthesis of 1-{3,4-difluoro-2-[2-fluoro-4-(2-triisopropylsilanyloxy-ethyl)-phenylamino]-phenyl}-2-triisopropylsilanyloxy-ethanol

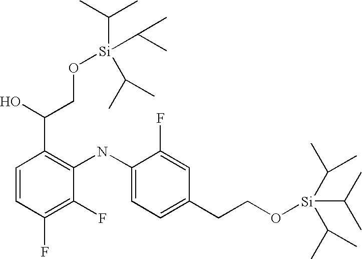

1-{3,4-Difluoro-2-[2-fluoro-4-(2-hydroxy-ethyl)-phenylamino]-phenyl}-2-triisopropylsilanyloxy-ethanol (1.35mmol) was protected as previously described in Example 6 step E. Product (520 mg, 60%, 2 steps) was obtained as a colorless oil after chromatography on silica gel eluting with 1:1 hexane/diethylether. MS (APCI+) 640 (M+1+); ¹H NMR(400 mHz, CDCl3) 7.15(m,1H), 6.94(m,2H), 6,78(m,1H), 6.47(m,1 H), 6.44(s,1 H), 4.87(m,1H), 3.82(t,2H), 3.72(m,2H), 2.76(t,2H), 1.1-0.90(m,42H).

### Step C: Synthesis of 1-{3,4-difluoro-2-[2-fluoro-4-(2-triisopropylsilanyloxy-ethyl)-phenylamino]-phenyl}-2-triisopropylsilanyloxy-ethanone

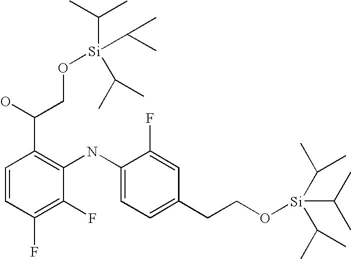

1-{3,4-Difluoro-2-[2-fluoro-4-(2-triisopropylsilanyloxy-ethyl)-phenylamino]-phenyl}-2-triisopropylsilanyloxy-ethanol (520 mg, 0.81 mmol) was oxidized employing the procedure in Example 6 step F to give an orange oil (364 mg) that was used directly in the next step to produce final product.

### Step D: Synthesis of 1-{3,4-difluoro-2-[2-fluoro-4-(2-hydroxy-ethyl)-phenylamino]-phenyl}-2-hydroxyl-ethanone

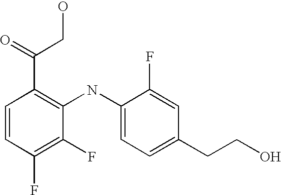

1-{3,4-Difluoro-2-[2-fluoro-4-(2-triisopropylsilanyloxy-ethyl)-phenylamino]-phenyl}-2-triisopropylsilanyloxy-ethanone (364 mg) was deprotected employing the procedure described in Example 7 step D to give product as a yellow solid (29 mg). MS (APCI−) 324 (M−1−); ¹H NMR(400 mHz, CDCl3) 9.90(s,1H), 7.43(m,1H), 7.07-6.92(m,3H), 6.70(m,1H), 4.83(s,2H), 3.86(t,2H), 2.84(t,2H).

### EXAMPLE 9

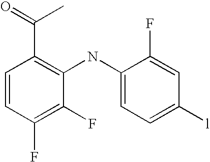

### 1-[3,4-difluoro-2-(2-fluoro-4-iodo-phenylamino)-phenyl]-ethanone

### Step A: Synthesis of 1-[3,4-difluoro-2-(2-fluoro-4-iodo-phenylamino)-phenyl]-ethanol

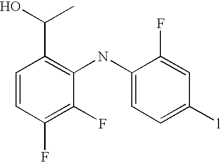

Hydroboration and oxidation of (2,3-Difluoro-6-vinyl-phenyl)-(2-fluoro-4-iodo-phenyl)-amine from Example 6 step C using the procedure described in Example 8 step A gave substantial amounts of the secondary alcohol that could be isolated by chromatography on silica gel eluting with 3:1 hexane/ethyl acetate. MS (APCI+) 394 (M+1+); ¹H NMR(400 mHz, CDCl3) 7.39(dd,1H), 7.28(m,1H), 7.08(dt,1H), 6.97(m,1H), 6.36(dt,1H), 4.98(q, 1H), 1.47(d,3H).

### Step B Synthesis of 1-[3,4-difluoro-2-(2-fluoro-4-iodo-phenylamino)-phenyl]-ethanone

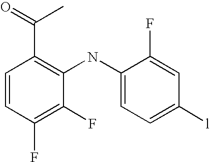

To 1-[3,4-difluoro-2-(2-fluoro-4-iodo-phenylamino)-phenyl]-ethanol (540 mg, 1.1 mmol) in acetone (10 mL) at 0° C. was added Jones reagent until the brown color persisted for 5 minutes. The reaction was then poured into water, extracted with ethyl acetate. The organic layer was washed with water and then brine to give a yellow oil. Chromatography on silica gel eluting with 2:1 hexane/dichloromethane provided product (120 mg, 22%). MS (APCI+) 392 (M+1+); ¹H NMR(400 mHz, CDCl3) 10.06(s,1H), 7.64(dq,1H), 7.42(dd,1H), 7.35(m,1H), 6.75(m,2H), 2.63(s,3H).

### EXAMPLE 10

### 1-[2-(4-Ethynyl-2-fluoro-phenylamino)-3,4-difluoro-phenyl]-ethanone

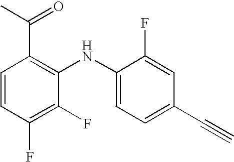

### Step A: Preparation of 1-[3,4-Difluoro-2-(2-fluoro-4-trimethylsilanylethynyl-phenylamino)-phenyl]-ethanone

3,4-Difluoro-2-(2-fluoro-4-trimethylsilanylethynyl-phenylamino)-N-methoxy-N-methyl-benzamide (1 g, 2.46 mmol) was dissolved in tetrahydrofuran (12 mL). Methylmagnesium bromide (4.01 mL, 1.4M in toluene/THF, 5.61 mmol) was added and the reaction stirred at room temperature for 1 hour. The reaction was quenched with saturated ammonium chloride solution, extracted with ethyl acetate, dried with MgSO₄, and concentrated. The product was taken on crude.

### Step B: Preparation of 1-[2-(4-Ethynyl-2-fluoro-phenylamino)-3,4-difluoro-phenyl]-ethanone

1-[3,4-Difluoro-2-(2-fluoro-4-trimethylsilanylethynyl-phenylamino)-phenyl]-ethanone (0.89 g, 2.46 mmol) was dissolved in tetrahydrofuran (10 mL). TBAF (2.71 mL, 1.0M in THF) was added and the reaction stirred at room temperature for 1 hour. Water was added and the mixture was extracted with ethyl acetate, dried with MgSO₄, and concentrated. The residue was purified on silica gel using a gradient of 18%-26% ethyl acetate in hexanes to yield the pure product (500 mg, 70% two steps). M.P.=78-80° C. 400 MHz ¹H-NMR (DMSO-d₆) δ 9.55 (s, 1H), 7.82 (m, 1H), 7.33 (dd, 1H, J=12.0, 1.7 Hz), 7.18 (m, 2H), 6.90 (m, 1H), 4.13 (s, 1H), 2.54 (s, 3H). MS (APCI+) 289.1 [M+1]. Anal. Calcd/Found for C₁₆H₁₀F₃NO: C, 66.44/66.29; H, 3.48/3.56; N, 4.84/4.76.

### EXAMPLE 11

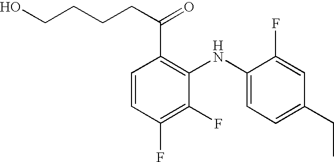

### 1-[2-(4-Ethyl-2-fluoro-phenylamino)-3,4-difluoro-phenyl]-5-hydroxy-pentan-1-one

### Step A: Preparation of 2-(4-Ethyl-2-fluoro-phenylamino)-3,4-difluoro-N-methoxy-N-methyl-benzamide

2-(4-Ethynyl-2-fluoro-phenylamino)-3,4-difluoro-N-methoxy-N-methyl-benzamide (4.11 g, 12.2 mmol) and 10% Pd/C (1.01 g) were taken up in tetrahydrofuran (100 mL) and stirred under and atmosphere of hydrogen for 16 hours. The mixture was filtered. The filtrate was concentrated to yield the product in near quantitative yield. 400 MHz ¹H-NMR (CDCl₃) δ 7.58 (s, 1H), 7.16 (m, 1H), 7.06 (m, 1H), 6.94 (dd, 1H, J=12.7, 1.7 Hz), 6.80 (d, 1H, J=8.3 Hz), 6.72 (m, 1 H), 3.35 (s, 3H), 3.03 (s, 3H), 2.47 (q, 2H, J=7.6 Hz), 1.08 (t, 3H, J=7.6 Hz).

### Step B: Preparation of 1-[2-(4-Ethyl-2-fluoro-phenylamino)-3,4-difluoro-phenyl]-pent-4-en-1-one

2-(4-Ethyl-2-fluoro-phenylamino)-3,4-difluoro-N-methoxy-N-methyl-benzamide (1.5 g, 4.43 mmol) was dissolved in THF. 3-Butenylmagnesium chloride (23.3 mL, 11.7 mmol) was added and the reaction stirred at room temperature for 30 minutes. Water was added and the mixture was extracted with ethyl acetate, dried with MgSO₄, and concentrated. The residue was purified on silica gel using a gradient of 0%-15% ethyl acetate in hexanes to yield the pure product (1.04 g, 70%). 400 MHz ¹H-NMR (CDCl₃) δ 9.66 (s, 1H), 7.86 (m, 1H), 7.02 (m, 2H), 6.92 (m, 2H), 5.78 (m, 1H), 4.99 (m, 1H), 4.90 (m, 1H), 3.10 (t, 2H, J=7.2 Hz), 2.52 (q, 2H, J=7.6 Hz), 2.28 (m, 2H), 1.11 (t, 3H, J=7.6 Hz).

### Step C: Preparation of 1-[2-(4-Ethyl-2-fluoro-phenylamino)-3,4-difluoro-phenyl]-5-hydroxy-pentan-1-one

1-[2-(4-Ethyl-2-fluoro-phenylamino)-3,4-difluoro-phenyl]-pent-4-en-1-one (0.83 g, 2.49 mmol) was cooled to 0° C. BH₃-THF (2.12 mL, 1.0 M in THF) was added and the reaction warmed to room temperature over 2 hours. Water (2 mL) and NaBO₃—H₂O (750 μL, 7.47 mmol) were added and the reaction continued to stir at room temperature for 1 hour. More water was added and the mixture was extracted with ethyl acetate, dried with MgSO₄, and concentrated. The residue was purified on silica gel using a gradient of 45%-50% ethyl acetate in hexanes to yield the pure product (240 mg, 27%). 400 MHz ¹H NMR (CDCl₃) δ 7.64 (m, 1 H), 6.92 (m, 3H), 6.66 (m, 1 H), 3.68 (t, 2H, J=6.4 Hz), 3.02 (t, 2H, J=7.1 Hz), 2.61 (q, 2H, J=7.6 Hz), 1.82 (m, 2H), 1.65 (m, 2H), 1.22 (t, 3H, J=7.6 Hz). MS (APCI+) 351.1 [M+1].

### EXAMPLE 12

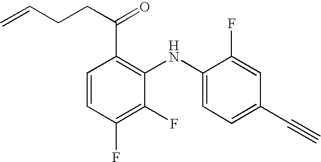

### 1-[2-(4-Ethynyl-2-fluoro-phenylamino)-3,4-difluoro-phenyl]-pent-4-en-1-one

2-(4-Ethynyl-2-fluoro-phenylamino)-3,4-difluoro-N-methoxy-N-methyl-benzamide (3.0 g, 8.97 mmol) was dissolved in tetrahydrofuran (50 mL) and cooled to −78° C. 3-Butenylmagnesium bromide (35.9 mL, 0.5 M in THF). The mixture stirred at −78° C. for 30 minutes, then warmed to room temperature overnight. The reaction was quenched with saturated ammonium chloride solution. The mixture was extracted with dichloromethane, dried with MgSO₄, and concentrated. The residue was purified on silica gel using a gradient of 5%-15% ethyl acetate in hexanes to yield the pure product (800 mg, 27%). 400 MHz ¹H-NMR (DMSO-d₆) δ 7.88 (m, 1H), 7.38 (dd, 1H, J=12.0, 2.0 Hz), 7.23 (m, 2H), 6.92 (m, 1H), 5.81 (m, 1H), 5.01 (dd, 1H, J=7.3, 2.0 Hz), 4.93 (dd, 1H, J=10.2, 2.0 Hz), 3.34 (s, 1H), 3.11 (t, 2H, J=7.3 Hz), 2.31 (m, 3H). MS (APCI+) 329.1 [M+1].

### EXAMPLE 13

Cellular Assay for Measuring MEK Inhibition

MEK inhibitors were evaluated by determining their ability to inhibit phosphorylation of MAP kinase (ERK) in murine colon 26 (C26) carcinoma cells. Since ERK1 and ERK2 represent the only known substrates for MEK1and MEK2, the measurement of inhibition of ERK phosphorylation in cells provides direct read out of cellular MEK inhibition by the compounds of the invention. Detection of phosphorylation of ERK was carried out either by Western blot or ELISA format. Briefly, the assays involve treatment of exponentially growing C26 cells with varying concentrations of the test compound (or vehicle control) for one hour at 37° C. For Western blot assay, cells were rinsed free of compound/vehicle and lysed in a solution containing 70 mM NaCl, 50 mM glycerol phosphate, 10 mM HEPES, pH 7.4, 1% Triton X-100, 1 mM Na₃VO₄, 100 μM PMSF, 10 μM leupeptin and 10 μM pepstatin. Supernatants were then subjected to gel electrophoresis and hybridized to a primary antibody recognizing dually phosphorylated ERK1 and ERK2. To evaluate total MAPK levels, blots were subsequently ‘stripped’ and re-probed with a 1:1 mixture of polyclonal antibodies recognizing unphosphorylated ERK1 and ERK2. For pERK ELISA assay, pERK TiterZyme Enzyme immunometric Assay kits were acquired from Assay Designs, Inc (Ann Arbor, Mich.). Briefly, cells were harvested in lysis solution containing 50 mM β-glycerophosphate, 10 mM HEPES, pH7.4, 70 mM NaCl, 2 mM EDTA and 1%SDS and protein lysates were diluted 1:15 with supplied Assay buffer prior to the execution of the assay. The subsequent steps were carried out essentially as recommended by the manufacturer.

The inhibition data generated by the above protocols is disclosed in Table I. If several concentrations of inhibitor were tested, IC₅₀ values (the concentration which gives 50% inhibition) were determined graphically from the dose response curve for % inhibition. Otherwise, percent inhibitions at measured concentrations are reported.

TABLE I
Cellular Inhibition of ERK Phosphorylation by
Compounds of the Invention

| Compound of Example No. | C26ELSA IC₅₀ (μM) | C26CPA1 IC₅₀ (μM) |
|---|---|---|
| 1 | 0.02 | 0.0065 |
| 2 | | 1 |
| 3 | | 1 |
| 4 | | 1 |
| 5 | | 0.287 |

### EXAMPLE 14

Carrageenan-Induced Footpad Edema (CFE) Rat Model

Male outbred Wistar rats (135-150 g, Charles River Labs) are dosed orally with 10 mL/kg vehicle or test compound 1 hour prior to administration of a sonicated suspension of carrageenan (1 mg/0.1 mL saline). Carrageenan is injected into the subplantar region of the right hind paw. Paw volume is determined by mercury plethysmography immediately after injection and again five hours after carrageenan injection. Percent inhibition of edema is determined, and the ID40 calculated by linear regression. Differences in swelling compared to control animals are assessed by a 1-way ANOVA, followed by Dunnett's test.

### EXAMPLE 15

Collagen-Induced Arthritis in Mice

Type II collagen-induced arthritis (CIA) in mice is an experimental model of arthritis that has a number of pathologic, immunologic, and genetic features in common with rheumatoid arthritis. The disease is induced by immunization of DBA/1 mice with 100 μg type II collagen, which is a major component of joint cartilage, delivered intradermally in Freund's complete adjuvant. The disease susceptibility is regulated by the class II MHC gene locus, which is analogous to the association of rheumatoid arthritis with HLA-DR4.

A progressive and inflammatory arthritis develops in the majority of mice immunized, characterized by paw width increases of up to 100%. A test compound is administered to mice in a range of amounts, such as 20, 60, 100, and 200 mg/kg body weight/day. The duration of the test can be several weeks to a few months, such as 40, 60, or 80 days. A clinical scoring index is used to assess disease progression from erythema and edema (stage 1), joint distortion (stage 2), to joint ankylosis (stage 3). The disease is variable in that it can affect one or all paws in an animal, resulting in a total possible score of 12 for each mouse. Histopathology of an arthritic joint reveals synovitis, pannus formation, and cartilage and bone erosions. All mouse strains that are susceptible to CIA are high antibody responders to type II collagen, and there is a marked cellular response to CII.

### EXAMPLE 16

SCW-Induced Monoarticular Arthritis

Arthritis is induced as described by Schwab et al., Infection and Immunity, 1991;59:4436-4442 with minor modifications. Rats receive 6 pg sonicated SCW [in 10 μL Dulbecco's PBS (DPBS)] by an intraarticular injection into the right tibiotalar joint on Day 0. On Day 21, the DTH is initiated with 100 μg of SCW (250 μL) administered IV. For oral compound studies, compounds are suspended in vehicle (0.5% hydroxypropyl-methylcellulose/0.2% Tween 80), sonicated, and administered twice daily (10 mL/kg volume) beginning 1 hour prior to reactivation with SCW. Compounds are administered in amounts between 10 and 500 mg/kg body weight/day, such as 20, 30, 60, 100, 200, and 300 mg/kg/day. Edema measurements are obtained by determining the baseline volumes of the sensitized hindpaw before reactivation on Day 21, and comparing them with volumes at subsequent time points such as Day 22, 23, 24, and 25. Paw volume is determined by mercury plethysmography.

### EXAMPLE 17

Mouse Ear-Heart Transplant Model

Fey T. A. et al. describe methods for transplanting split-heart neonatal cardiac grafts into the ear pinna of mice and rats (J. Pharm. and Toxic. Meth., 1998;39:9-17). Compounds are dissolved in solutions containing combinations of absolute ethanol, 0.2% hydroxypropyl methylcellulose in water, propylene glycol, cremophor, and dextrose, or other solvent or suspending vehicle. Mice are dosed orally or intraperitoneally once, twice or three times daily from the day of transplant (Day 0) through Day 13 or until grafts have been rejected. Rats are dosed once, twice, or three times daily from Day 0 through Day 13. Each animal is anesthetized and an incision is made at the base of the recipient ear, cutting only the dorsal epidermis and dermis. The incision is spread open and down to the cartilage parallel to the head, and sufficiently wide to accommodate the appropriate tunneling for a rat or insertion tool for a mouse. A neonatal mouse or rat pup less than 60 hours old is anesthetized and cervically dislocated. The heart is removed from the chest, rinsed with saline, bisected longitudinally with a scalpel, and rinsed with sterile saline. The donor heart fragment is placed into the preformed tunnel with the insertion tool and air or residual fluid is gently expressed from the tunnel with light pressure. No suturing, adhesive bonding, bandaging, or treatment with antibiotics is required.

Implants are examined at 10- to 20-fold magnification with a stereoscopic dissecting microscope without anesthesia. Recipients whose grafts are not visibly beating may be anesthetized and evaluated for the presence of electrical activity using Grass E-2 platinum subdermal pin microelectodes placed either in the pinna or directly into the graft and a tachograph. Implants can be examined 1 to 4 times a day for 10, 20, 30 or more days. The ability of a test compound to ameliorate symptoms of transplant rejection can be compared with a control compound such as cyclosporine, tacrolimus, or orally-administered lefluonomide.

### EXAMPLE 18

The analgesic activity of the compounds of the present invention is assessed by a test with rats. Rats weighing from 175 to 200 g are injected with carrageenan (2% in 0.9% sodium chloride aqueous solution, 100 μL injection volume) into the footpad of one hind limb. The rats are placed on a glass plate with illumination from a halogen lamp placed directly under the injected paw. The time (in seconds) from beginning illumination until the hind limb was withdrawn from the glass was measured and scored as Paw Withdrawal Latency (PWL). Drug substances were given by oral gavage injection 2½ hours after carrageenan injection to the footpad. PWL was measured prior to carrageenan injection, just prior to drug injection, and 1, 2 (and sometimes 3) hours after drug injection.

Carrageenan (a polysaccharide extracted from seaweed) causes a sterile inflammation when injected under the skin. Injection into the rat footpad causes little or no spontaneous pain-related behavior but induces hyperalgesia (pain-related behavioral responses of greater intensity than expected) to peripheral thermal or mechanical stimuli. This hyperalgesia is maximal 2 to 3 hours after injection. Treatment of rats with various analgesic drugs reduces hyperalgesia measured in this way and is a conventional test for detection of analgesic activity in rats. (Hargreaves K, Dubner R, Brown F, Flores C, Joris J. A new and sensitive method for measuring thermal nociception in cutaneous hyperalgesia. Pain, 1988;32:77-88 and Kayser V, Guilbaud G. Local and remote modifications of nociceptive sensitivity during carrageenan-induced inflammation in the rat. Pain, 1987;28:99-108). Untreated rats have a PWL of approximately 10 seconds. Carrageenan injection reduces PWL to approximately 3 seconds for at least 4 hours, indicating thermal hyperalgesia. Inhibition of the carrageenan thermal hyperalgesia response is determined by the difference between reduced PWL prior to drug and subsequent to drug treatment, and was expressed as percent inhibition of the response. Administration of MEK inhibitors dose-dependently reduced thermal hyperalgesia.

1. A compound of Formula I 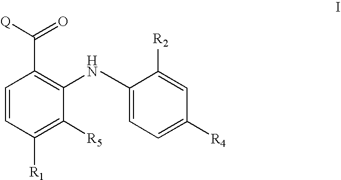
wherein
Q is C₁₋₆ alkyl, C₂₋₆ alkenyl, C₂₋₆ alkynyl, or C₂₋₅ heteroaryl, wherein the C₁₋₆ alkyl, C₂₋₆ alkenyl, and C₂₋₆ alkynyl groups are optionally substituted with between 1 and 2 hydroxy substituents, and the C₂₋₅ heteroaryl is optionally substituted with C₁₋₄ alkyl, and further wherein the C₁₋₄ alkyl is optionally substituted with between 1 and 3 substituents independently selected from hydroxy and amino;
R₁ and R₅ are each independently hydrogen or fluorine;
R₂ is hydrogen, chlorine, fluorine or methyl;
R₄ is bromine, chlorine, fluorine, iodine, C₁₋₆ alkyl, C₂₋₆ alkenyl, C₂₋₆ alkynyl, C₃₋₆ cycloalkyl, —(CH₂)—C₃₋₆ cycloalkyl, cyano, —O—(C₁₋₄ alkyl), —S—(C₁₋₂ alkyl), —SOCH₃, —SO₂CH₃, —SO₂NR₆R₇, —C≡C—(CH₂)ₙNH₂, —C≡C—(CH₂)ₙNHCH₃, —C≡C—(CH₂)ₙN(CH₃)₂, —C≡C—CH₂OCH₃, —C═C(CH₂)ₙOH, —C═C—(CH₂)ₙNH₂, (Z)-CHCHCH₂OCH₃, -(Z)-CHCH—(CH₂)ₙNHCH₃, (Z)-CHCH—(CH₂)ₙN(CH₃)₂, —(CH₂)ₚCO₂R₆, C(O)C₁₋₃ alkyl, C(O)NHCH₃, —(CH₂)ₘNH₂, —(CH₂)ₘNHCH₃, —(CH₂)ₘN(CH₃)₂, —(CH₂)ₘOR₆, —CH₂S(CH₂)ₜ(CH₃), —(CH₂)ₚCF₃, —C≡CCF₃, —CH═CHCF₃, —CH₂CHCF₂, —CH═CF₂, —(CF₂)ᵥCF₃, —CH₂(CF₂)ₙCF₃, —(CH₂)ₜCF(CF₃)₂, —CH(CF₃)₂, —CF₂CF(CF₃)₂, or —C(CF₃)₃, wherein the C₁₋₆ alkyl and C₂₋₆ alkynyl are optionally substituted with between 1 and 3 substituents independently selected from hydroxy and alkyl;
R₆ and R₇ are each independently hydrogen, methyl, or ethyl;
m is 1 to 4;
n is 1 to 2;
p is 0 to 2;
t is 0 to 1;
v is 1 to 5;
and pharmaceutically acceptable salts, C₁₋₆ amides and C₁₋₆ esters thereof.

2. The compound of claim 1 wherein Q is C₁₋₆ alkyl, C₂₋₆ alkenyl, or C₂₋₅ heteroaryl, wherein the C₁₋₆ alkyl group is optionally substituted with between 1 and 2 hydroxy substituents and the C₂₋₅ heteroaryl is optionally substituted with methyl.

3. The compound of claim 1, wherein Q is C₂₋₅ heteroaryl containing from one to two heteroatoms independently selected from the group consisting of nitrogen, oxygen and sulfur, and further wherein the C₂₋₅ heteroaryl is optionally substituted with C₁₋₄ alkyl, and further wherein the C₁₋₄ alkyl is optionally substituted with between 1 and 3 substituents independently selected from hydroxy and amino.

4. The compound of claim 1, wherein Q is methyl, —CH₂OH, —(CH₂)₄OH, butenyl, methyl-substituted oxazolyl, thiazolyl, methyl-substituted imidazolyl, or pyridinyl.

5. The compound of claim 1 wherein R₁ is fluorine.

6. The compound of claim 1 wherein R₅ is fluorine.

7. The compound of claim 1 wherein R₂ is fluorine.

8. The compound of claim 1 wherein R₄ is iodine, C₁₋₆ alkyl, C₂-₆ alkenyl, or C₂₋₆ alkynyl, and further wherein the C₁₋₆ alkyl, C₂₋₆ alkenyl, and C₂₋₆ alkynyl are optionally substituted with hydroxy.

9. The compound of claim 1 which is
1-[3,4-Difluoro-2-(2-fluoro-4-iodo-phenylamino)-phenyl]-2-hydroxy-ethanone;
[2-(4-Ethynyl-2-fluoro-phenylamino)-3,4-difluoro-phenyl]-pyridin-2-yl-methanone;
[2-(4-Ethynyl-2-fluoro-phenylamino)-3,4-difluoro-phenyl]-thiazol-2-yl-methanone;
[2-(4-Ethynyl-2-fluoro-phenylamino)-3,4-difluoro-phenyl]-(1-methyl-1H-imidazol-2-yl)-methanone;
[3,4-Difluoro-2-(2-fluoro-4-iodo-phenylamino)-phenyl]-(4-methyl-oxazol-2-yl)-methanone;
1-[3,4-Difluoro-2-(2-fluoro-4-iodo-phenylamino)-phenyl]-ethanone;
1-{3,4-Difluoro-2-[2-fluoro-4-(2-hydroxy-ethyl)-phenylamino]-phenyl}-2-hydroxy-ethanone;
1-[2-(4-Ethyl-2-fluoro-phenylamino)-3,4-difluoro-phenyl]-2-hydroxy-ethanone;
1-[2-(4-Ethynyl-2-fluoro-phenylamino)-3,4-difluoro-phenyl]-ethanone;
1-[2-(4-Ethyl-2-fluoro-phenylamino)-3,4-difluoro-phenyl]-5-hydroxy-pentan-1-one;
1-[3,4-Difluoro-2-(2-fluoro-4-vinyl-phenylamino)-phenyl]-2-hydroxy-ethanone; or
1-[2-(4-Ethynyl-2-fluoro-phenylamino)-3,4-difluoro-phenyl]-pent-4-en-1-one.

10. A pharmaceutical composition comprising a compound of claim 1 and a pharmaceutically acceptable carrier.

11. A method of treating a proliferative disease in a patient in need thereof comprising administering a therapeutically effective amount of a compound of claim 1.

12. A method of treating cancer in a patient in need thereof comprising administering a therapeutically effective amount of a compound of claim 1.

13. A method of treating psoriasis in a patient in need thereof comprising administering a therapeutically effective amount of a compound of claim 1.

14. A method of treating restenosis, autoimmune disease, atherosclerosis, rheumatoid arthritis, heart failure, chronic pain, neuropathic pain, or osteoarthritis in a patient in need thereof comprising administering a therapeutically effective amount of a compound of claim 1.

15. A method for treating cancer in a patient in need thereof comprising administering a therapeutically effective amount of a compound of claim 1 in combination with radiation therapy or at least one chemotherapeutic agent.
